(19) United States
(12) Patent Application Publication
(10) Pub. No.: US 2024/0391889 A1
(43) Pub. Date: Nov. 28, 2024

(54) ANTIMICROBIAL COMPOUNDS AND THE METHODS OF USE

(71) Applicant: Middle Tennessee State University, Murfreesboro, TN (US)

(72) Inventors: Kevin Lee Bicker, Rockvale, TN (US); Robert Madison Green, Nashville, TN (US)

(21) Appl. No.: 18/695,490

(22) PCT Filed: Sep. 30, 2022
(86) PCT No.: PCT/US2022/045350

§ 371 Date: Mar. 26, 2024

Related U.S. Application Data
(60) Provisional application No. 63/251,401, filed on Oct. 1, 2021.

(51) Int. Cl. C07D 307/36; A61K 31/341; A61P 31/04; A61P 31/10

(52) U.S. Cl. CPC C07D 307/36; A61K 31/341; A61P 31/04; A61P 31/10

(57) ABSTRACT

Peptoids, compositions thereof, and methods of using the peptoids. The peptoid may be an antimicrobial peptoid. In some embodiments, the peptoid may be used to treat or prevent a fungal infection, a biofilm produced from a fungal infection, or a combination thereof, in a vertebrate, such as a human or an animal, or in a plant. In some embodiments, the peptoid may be used to treat or prevent a bacterial infection, or biofilm produced from a bacterial infection, or a combination thereof, in a vertebrate, such as a human or an animal, or in a plant.

### CROSS-REFERENCE TO RELATED APPLICATION

This application claims the benefit of U.S. Provisional Patent Application No. 63/251,401, filed Oct. 1, 2021, which is incorporated herein by reference in its entirety.

### GOVERNMENT FUNDING

This invention was made with government support under 1R03A1146393-01A1 awarded by the National Institutes of Health. The government has certain rights in the invention.

### FIELD

The present disclosure generally relates to peptoid, more specifically, antimicrobial peptoid compounds; compositions containing the same; and methods of using the compounds and/or compositions.

### INTRODUCTION

Candida species are the leading cause of fungal hospital acquired infections (HAIs) with over 7 million cases per year and a mortality rate of 33% (Pfaller et al., Clin. Microbiol. Rev. 20, 133-163 (2007)). C. albicans is commonly found on the body or in the mouth and generally does not result in health problems for the host unless there is a change in environment such as pH or nutrient availability. Candidemia, which is a C. albicans infection of the blood stream, has a mortality rate of 40-60% in healthy individuals (Sarma et al., Infect. Drug Resist. 10, 155-165 (2017)). C. albicans can switch their morphology between budding yeast, pseudo hyphae, or hyphae depending on the environment (Sudbery et al., Nat. Rev. Microbiol. 9, 737 (2011)). In nutrient rich environments, C. albicans are primarily budding yeasts, but as the environment becomes more unfavorable, they switch to a more robust hyphae morphology. In the hyphae morphology, C. albicans secrete polysaccharides and other biomolecules that form a 3D matrix termed a biofilm (Douglas et al., Trends Microbiol. 11, 30-36 (2003)). Biofilms allow pathogens to grow on abiotic surfaces, such as counters, catheters, implanted prostheses (Ramage et al., FEMS Yeast Res. 6, 979-986 (2006)), and ventilators (Nobile et al., Rev. Microbiol. 69, 71-92 (2015)). Biofilms include polysaccharides, proteins, extracellular DNA, and small molecules that are used for protection and quorum sensing (QS) (Sztajer et al., ISMEJ 8, 2256-2271 (2014)). The polysaccharide mixture makes it difficult for therapeutics to reach the pathogen, thus requiring alternative treatments to combat the pathogen.

Biofilms can include a single species, multiple species, or even pathogens from different kingdoms (Sztajer et al., ISME J. 8, 2256-2271 (2014)). Opportunistic bacteria can embed themselves into fungal biofilms to create a cross-kingdom biofilm. In these scenarios, the patient not only has to be treated for the initial infection but requires multiple antibiotics to combat the more complex cross-kingdom infection. For example, there have been documented cases of S. aureus being found in a C. albicans biofilm (Harriott et al., Antimicrob. Agents Chemother. 53, 3914-3922 (2009)). Both species can release QS molecules to signal for either species to proliferate or slow down production of compounds. Farnesol is a QS molecule that stimulates hyphae formation released by most fungal species, including C. neoformans (Peleg et al., Proc. Natl. Acad. Sci. 105, 14585-14590 (2008)). Importantly, not all cross-kingdom biofilms are synergistic. A. baumannii will inhibit growth of C. albicans biofilm during initial growth, but once the biofilm is established C. albicans will inhibit growth of A. baumannii (Kostoulias et al., Antimicrob. Agents Chemother. 60, 161-167 (2016)). C. albicans can do this through the release of farnesol, a small molecule shown to inhibit A. baumannii growth.

### SUMMARY OF THE APPLICATION

The development of an antimicrobial agent that not only targets planktonically growing microbes but also targets the pathogen's biofilm would be very advantageous in treating HAIs. This disclosure describes peptoids, compositions thereof, and methods of using the peptoids to treat and/or prevent fungal and/or bacterial infections and/or biofilms produced from bacterial and/or fungal infections, in a vertebrate or a plant.

In one aspect, this disclosure describes a compound of the general formula

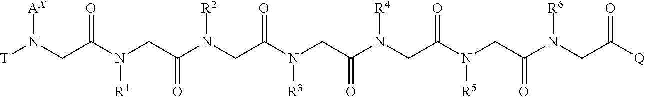

a protonated form thereof, a pharmaceutically acceptable salt thereof, or both. A^{X} is H or a linear or branched (C₆ to C₂₀)alkyl or a linear or branched (C₆ to C₂₀)alkenyl, wherein the alkyl or the alkenyl optionally includes a carbonyl group. T may be a linear or branched (C₆ to C₂₀)alkyl or a linear or branched (C₆ to C₂₀)alkenyl, wherein the alkyl or the alkenyl optionally includes a carbonyl group. Q may be a hydroxyl or NH₂. R¹, R², R³, R⁴, R⁵, and R⁶ are each independently

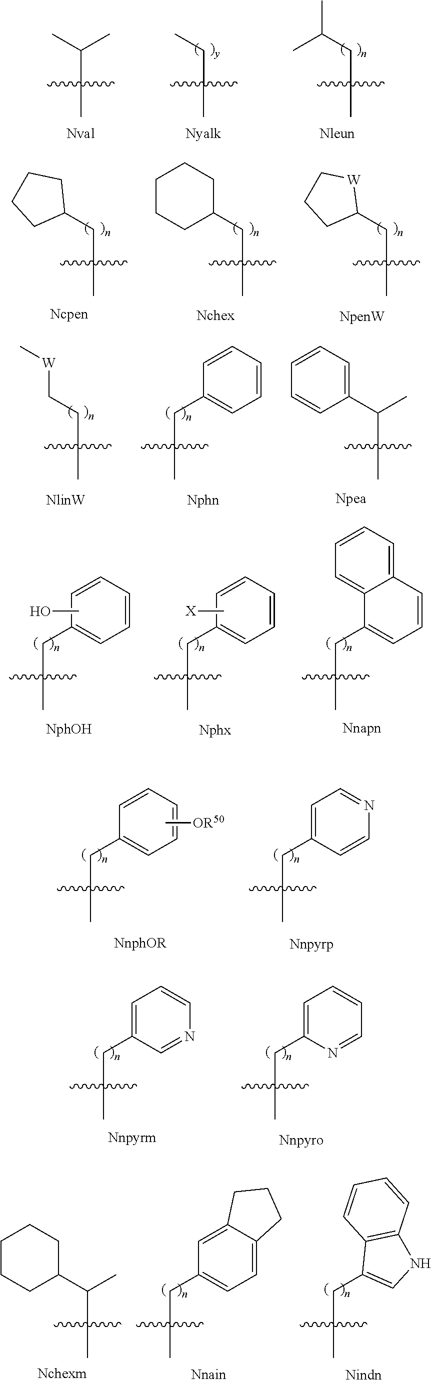

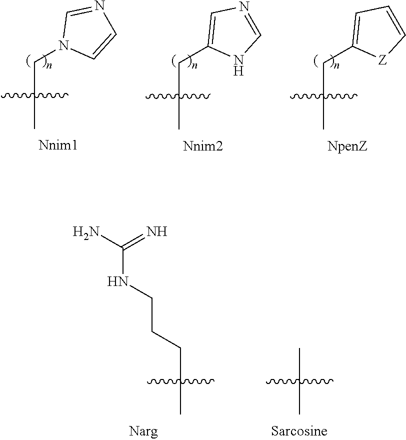

or an alkyl amine of the general formula R¹⁰NR¹¹R¹²R¹³. Each n is independently (C₀ to C₃)alkylene. Each y is independently (C₀ to C₆)alkylene. For each R¹, R², R³, R⁴, R⁵, and/or R⁶ that is NcpenW, each W is independently N, S, or O. For each R¹, R², R³, R⁴, R⁵, and/or R⁶ that is NlinW, each W is independently N, S, or O. For each R¹, R², R³, R⁴, R⁵, and/or R⁶ that is NphX, each X is independently F, Cl, Br, or I. For each R¹, R², R³, R⁴, R⁵, and/or R⁶ that is NpenZ, each Z is independently S, or O. For each R¹, R², R³, R⁴, R⁵, and/or R⁶ that is R¹⁰NR¹¹R¹²R¹³, each R¹⁰ is independently a linear (C₁ to C₆)alkylene. For each R¹, R², R³, R⁴, R⁵, and/or R⁶ that is R¹⁰NR¹¹R¹²R¹³, each R¹¹, each R¹², and each R¹³ are independently H or (C₁ to C₆)alkyl.

In another aspect, this disclosure describes a composition that include a compound of the previous aspect and a pharmaceutically acceptable carrier or a fungicidal acceptable carrier.

In another aspect, this disclosure describes method of administering the compound and/or composition of anyone of the preceding aspects to a subject to prevent and/or treat a fungal infection, a bacterial infection, a biofilm produced from a fungal infection, a biofilm produced from a bacterial infection, or combinations thereof. In some embodiments, the subject is a vertebrate such as human, domestic animal, or companion animal. In some embodiments, the subject is a plant.

### DEFINITIONS

Terms used herein will be understood to take on their ordinary meaning in the relevant art unless specified otherwise. Several terms used herein, and their meanings are set forth below.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

As used herein, “providing” in the context of a composition or a compound means making the composition or compound, purchasing the composition or compound, or otherwise obtaining the composition or compound.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features (e.g., chemical functional groups such as T, A^{X}, Q, R¹, R², R³, R⁴, R⁵, and R⁶), configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7.3, 4, 5, 5.3, and 6. This applies regardless of the breadth of the range.

As used herein, “aliphatic group” means a saturated or unsaturated linear or branched hydrocarbon group. This term is used to encompass alkyl, alkenyl, alkylene, and alkynyl groups, for example.

As used herein, “alkyl” refers to a monovalent group that is a radical of an alkane and includes straight-chain, branched-chain, cyclic, and bicyclic alkyl groups, and combinations thereof, including both unsubstituted and substituted alkyl groups.

The term “alkylene” refers to a divalent group that is a radical of an alkane and includes groups that are linear, branched, cyclic, bicyclic, or a combination thereof.

The term “alkenyl” refers to a univalent group that is a radical of an alkene and includes groups that are linear, branched, cyclic, or combinations thereof. An alkenyl group has one or more double bonds. The location of the double bond may be anywhere along the alkenyl.

The term “aromatic” refers to a cyclic, fully conjugated planar structure that obeys Hückel's rules, that is, the compound has 4n+2π electrons where n is a positive integer or zero. For example, benzene has 6π electrons. Thus, 6=4n+2π. Solving for n gives 1. Therefore, benzene is an aromatic compound.

The term “backbone” refers to the longest contiguous chain. One or more branches may be covalently bonded to the backbone.

As used herein, the term “infection” refers to the presence of and multiplication of a microbe in the body of a subject. The infection can be clinically in apparent or result in symptoms associated with disease caused by the microbe. The infection can be at an early stage, or at a late stage. Examples of a microbe include a fungus and a bacterium.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1 shows a diagram of the individual stages for the Inverted Peptoid Library Agar Diffusion (iPLAD) assay. Stage 1: A mature C. albicans biofilm is formed on a tissue-culture treated Petri dish. Stage 2: PLAD linked peptoid library beads are embedded in soft agar containing a reducing agent on top of the established biofilm. Overnight incubation allows for release of the β-strand peptoid from the bead to interact with fungi. Stage 3: The cell viability indicator phloxine B is added and diffuses through the agar. If the peptoid released from a bead is toxic to the fungi, then those fungi are unable to pump out phloxine B, staining them a reddish color. Stage 4: Hit beads are removed and the α-strand peptoid is cleaved for structure determination by tandem mass spectrometry.

FIG. 2A shows the structure of RGL9 on the solid-phase PLAD linker system. Positions C contain cationic submonomers and positions R include aromatic or aliphatic submonomers.

FIG. 2B shows examples of possible cationic (C) and aromatic/aliphatic (R) side chain groups that may be incorporated into RGL9.

FIG. 2C shows the structure of RMG9-11, a peptoid identified from an iPLAD screening campaign against C. albicans.

FIG. 3 show the structures of RMG9-11 derivatives.

FIG. 4 shows the mass spectrum of RMG9-11.

FIG. 5 shows the sarcosine RMG9-11 derivatives tested in the sarcosine scan.

FIG. 6 show the structures of the RMG9-11 derivates tested in the first round of structure activity relationship (SAR) studies.

FIG. 7 shows the structures of SAR round 1 hybrid compounds.

FIG. 8 show the structures of the RMG9-11 derivates tested in the second round of SAR studies

FIG. 9 shows the structures of the contemplated SAR round 2 hybrid compounds.

FIG. 10 show the structures of the contemplated RMG9-11 derivates for a third structure activity relationship (SAR) study.

### DETAILED DESCRIPTION

### Peptoids

Described herein are peptoids, compositions thereof, and methods of making and using the peptoids. In some embodiments, the peptoids of the present disclosure are antimicrobial peptoids. In some embodiments, compositions including a peptoid of the present disclosure may be administered to a subject infected with, or at risk of being infected with a pathogenic microbe, a biofilm produced from infection with a pathogenic microbe, or a combination thereof.

In some embodiments, compositions including the peptoid may be administered to a subject infected with, or at risk of being infected with pathogenic fungus, or a biofilm produced from infection with a pathogenic fungus, or a combination thereof, including, for example, infection with a Candida spp. including, for example, C. albicans, C. tropicalis, C. stellatoidea, C. glabrata, C. krusei, C. parapsilosis, C. guilliermondii, C. viswanathii, C. auris, or C. lusitaniae, or a combination thereof; an infection with Rhodotorula spp., including, for example, R. mucilaginosa, R. minuta, or R. glutinis, or a combination thereof; and/or an infection with Cryptococcus spp. for example, C. neoformans or Cryptococcus gattii, or a combination thereof.

In some embodiments, compositions including the peptoid may be administered to a subject infected with or at risk of being infected with pathogenic bacterium, or a biofilm produced from infection with a pathogenic bacterium, or a combination thereof, including, for example Enterococcus faecium, Staphylococcus aureus, Enterococcus Pseudomonas aeruginosa, Enterobacter, Klebsiella pneumoniae, Escherichia coli, Acinetobacter baumannii, or Mycobacterium tuberculosis, or a combination thereof.

### Antimicrobial Peptides and Peptoids

Many organisms use antimicrobial peptides (AMPs) as a part of their innate immune response against pathogenic bacteria and fungi. (Zasloff et al., Nature 2002, 415(6870), 389-395). AMPs may be advantageous as clinical antimicrobial agents given their relatively high specificity for microorganisms over mammalian cells and lack of observed drug resistance, likely stemming from their generally accepted mode of action. Most AMPs used against fungi target the cell membrane, forming pores or causing changes in cell permeability that result in a leakage of cytoplasmic components, ultimately resulting in pathogen death (Zasloff et al., Nature 2002, 415(6870), 389-395). These AMPs are able to take advantage of key differences between mammalian and fungal cell membranes, such as ergosterol, a principal sterol only present in fungal cell membranes, to selectively target the pathogen (Walsh et al., Clin. Infect. Dis. 2008, 46 (3), 327-360). Additionally, AMPs have been shown to prevent biofilm formation and kill pathogens within an established biofilm (Yasir et al., Materials 11, (2018); Galdiero et al., Pharmaceutics 11, 322 (2019)). The mechanism of action for these anti-biofilm peptides is generally membrane disruption, though interference in cell signaling systems or breakdown of the biofilm itself has also been observed (Yasir et al., Materials 11, (2018)). However, peptides are readily recognized and degraded by proteases and are often poor clinical therapeutics for combating in vivo fungal infections (Latham et al., Nat. Biotech. 1999, 17(8), 755-757; Zhang et al., Expert Opin. Pharmacother. 2006, 7(6), 653-663).

One means of resolving the proteolytic instability of AMPS involves the use of N-substituted glycines, also referred to as peptoids. In peptides the side chain R group is attached to the α-carbon, whereas in peptoids the R group is attached to the amide nitrogen. This alteration in structure makes peptoids unrecognizable by proteases while maintaining many of the advantageous properties of peptides and extending in vivo half-life (Culf et al. Molecules 2010, 15(8), 5282-5335; Zuckermann et al. J. Am. Chem. Soc. 1992, 114(26), 10646-10647).

### Peptoids

In one aspect, this disclosure describes a peptoid of the general Formula I:

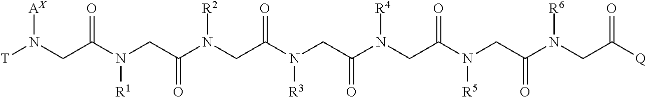

a protonated form thereof, a pharmaceutical acceptable salt thereof, or both. In some embodiments, the peptoids of the general Formula I have antimicrobial properties and are therefor antimicrobial peptoids.

As used herein, the term “a protonated form thereof” refers to a peptoid, an R group (R¹, R², R³, R⁴, R⁵, or R⁶), a T group, an A^{X} group, or a Q group that is protonated at certain pH values (e.g., biological pH), and therefore, possesses a formal charge. Multiple R groups may be protonated and therefore, the formal charge may be greater than +1 (e.g., 2, +3, +4, etc.) If one or more of an R group (R¹, R², R³, R⁴, R⁵, or R⁶), a T group, a A^{X} group, or a Q group is protonated, the entire peptoid may be referred to as protonated (i.e., a protonated form of a peptoid).

Formula I is a peptoid with six N-substituted glycine repeats. Each repeat has one side chain (R¹, R², R³, R⁴, R⁵, or R⁶) that is covalently bonded to the amide nitrogen of the peptoid backbone. The peptoid has an N-terminal cap group T, an optional second N-terminal cap group A^{X} (e.g., A^{X} may be H or an N-terminal cap group T), and a C-terminal cap group Q.

Generally, T is hydrophobic. T may be a linear or branched alkyl, or a linear or branched alkenyl. The alkyl or alkenyl may optionally include a carbonyl. Preferably, a carbonyl-containing alkyl or alkenyl group is of the general formula (CO)—R²⁰, where R²⁰ is the alkyl or alkenyl group. In some embodiments, R²⁰ is a linear alkyl of at least C₆, at least C₁₀, or at least C₁₅. In some embodiments, R²⁰ is a linear alkyl no greater than C₂₀, no greater than C₁₅, or no greater than C₁₀. In some embodiments, R²⁰ is (C₆ to C₂₀)alkyl, (C₆ to C₁₅)alkyl, or (C₆ to C₂₀)alkyl. In some embodiments, R²⁰ is (C₁₀ to C₂₀)alkyl or (C₁₅ to C₂₀)alkyl. In some embodiments, R²⁰ is (C₁₀ to C₁₅)alkyl. In some embodiments, R²⁰ is a linear alkyl selected from n-hexyl, n-heptyl, n-octyl, n-nonyl, n-decyl, n-undecyl, n-dodecyl, n-tridecyl, n-tetradecyl, n-pentadactyl, n-hexadecyl, n-heptadecyl, n-octadecyl, n-nonadecyl, and n-eicosanyl. In some embodiments, T has the general formula (CO)—R²⁰ is linear (C₁₅)alkyl. In some embodiments, T has the general formula (OC)—R²⁰ where R²⁰ is linear (C₁₃)alkyl. In some embodiments, T is derived from palmitic acid. In such embodiments, T has the general formula (OC)—R²⁰ where R²⁰ is C₁₅H₂₇. In some embodiments, T is derived from myristic acid. In some such embodiments, T has the general formula (OC)—R²⁰ where R²⁰ is C₁₃H₃₁.

In some embodiments, T is a linear alkyl of at least C₆, at least C₁₀, or at least C₁₅. In some embodiments, T is a linear alkyl no greater than Cao, no greater than C₁₅, or no greater than C₁₀. In some embodiments, T is a linear (C₆ to C₂₀)alkyl, a linear (C₆ to C₁₅)alkyl, or a linear (C₆ to C₁₀)alkyl. In some embodiments, T is a linear (C₁₀ to C₂₀)alkyl or a linear (C₁₅ to C₂₀)alkyl. In some embodiments, T is a linear (C₁₀ to C₁₅)alkyl. In some embodiments, T is n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosanyl. According to some embodiments, T is n-tridecyl (linear (C₁₃)alkyl; C₁₃H₂₇; sometimes called Ntri). According to some embodiments, T is n-octyl (linear (C₈)alkyl; C₈H₁₇). According to some embodiments, T is n-hexyl (linear (C₆)alkyl; C₆H₁₃). According to some embodiments, T is n-pentadecyl (linear (C₁₅)alkyl; C₁₅H₃₁).

In some embodiments, T is a branched alkyl. The branched alkyl has a backbone and one or more branches that are covalently bonded to the backbone. In some embodiments, the backbone is an alkyl of at least C₆, at least C₁₀, or at least C₁₅. In some embodiments, the backbone is an alkyl that is no greater than C₂₀, no greater than C₁₅, or no greater than C₁₀. In some embodiments the backbone is (C₆ to C₂₀)alkyl, (C₆ to C₁₅)alkyl, or (C₆ to C₁₀)alkyl. In some embodiments, the backbone is (C₁₀ to C₂₀)alkyl or (C₁₀ to C₁₅)alkyl. In some embodiments, the backbone is (C₁₅ to C₂₀)alkyl.

One or more alkyl branches are covalently bonded to the backbone of a branched alkyl. In some embodiments, the branch is an alkyl of at least C₁, at least C₅, or at least C₁₀. In some embodiments, the branch is an alkyl that is no greater than C₁₀, no greater than C₅, or no greater than C₁. In some embodiments, the branch is (C₁ to C₁₀)alkyl or (C₁ to C₅)alkyl. In some embodiments, the branch is (C₅ to C₁₀)alkyl. In some embodiments, the alkyl branch of the branched alkyl is methyl, ethyl, propyl, butyl, pentyl, hexyl, heptyl, octyl, nonyl, decyl, undecyl, or dodecyl. In some embodiments, the branched alkyl has a single alkyl branch that is covalently bonded to the backbone. In some embodiments, the branched alkyl has two or more branches covalently bonded to the backbone.

In some embodiments, T is a linear alkenyl. In some embodiments, T is a linear alkenyl of at least C₆, at least C₁₀, or at least C₁₅. In some embodiments, T is a linear alkenyl no greater than C₂₀, no greater than C₁₅, or no greater than C₁₀. In some embodiments, T is a linear (C₆ to C₂₀)alkenyl, a linear (C₆ to C₁₅)alkenyl, or a linear (C₆ to C₁₀)alkenyl. In some embodiments, T is a linear (C₁₀ to C₂₀)alkenyl or a linear (C₁₀ to C₁₅)alkenyl. In some embodiments, T is a linear (C₁₅ to C₂₀)alkenyl. In some embodiments, T is n-hexenyl, n-heptenyl, n-octenyl, n-nonenyl, n-decenyl, n-undecenyl, n-dodecenyl, n-tridecenyl, n-tetradecenyl, n-pentadacenyl, n-hexadecenyl, n-heptadecenyl, n-octadecenyl, nnonadecenyl, and n-eicosenyl. In some embodiments, the linear alkenyl may have a single double bond. In some embodiments, the linear alkenyl may have two or more double bonds. The maximum degree of unsaturation, and therefore the maximum number of double bonds for a linear alkenyl where the total number of carbons (Cₙ) is an even number, is Cₙ/2. The maximum degree of unsaturation for a linear alkenyl where the total number of carbons (Cₙ) is an odd number, is (Cₙ−1)/2. In embodiments where the linear alkenyl has two or more double bonds, the double bonds may be positioned at any location along the alkenyl.

In some embodiments, T is a branched alkenyl. The branched alkenyl has a backbone and one or more branches that are covalently bonded to the backbone. In some embodiments, the backbone is an alkenyl of at least C₆, at least C₁₀, or at least C₁₅. In some embodiments, the backbone is an alkenyl that is no greater than C₂₀, no greater than C₁₅, or no greater than C₁₀. In some embodiments the backbone is (C₆ to C₂₀)alkenyl, (C₆ to C₁₅)alkenyl, or (C₆ to C₁₀)alkenyl. In some embodiments, the backbone is (C₁₀ to C₂₀)alkenyl or (C₁₀ to C₁₅)alkenyl. In some embodiments, the backbone is (C₁₅ to C₂₀)alkenyl. In some embodiments, the backbone is (C₈)alkenyl. In some embodiments, the backbone is (C₁₂)alkenyl. In some embodiments, the alkenyl backbone of the branched alkenyl may have a single double bond. In some embodiments, the alkenyl backbone of the branched alkenyl has two or more double bonds. The maximum degree of unsaturation, and therefore the maximum number of double bonds, for an alkenyl backbone where the total number of carbons (Cₙ), is an even number is Cₙ/2. The maximum degree of unsaturation for an alkenyl backbone where the total number of carbons (Cₙ) is an odd number, is (Cₙ−1)/2. In embodiments where the alkenyl backbone of the branched alkenyl has two or more double bonds, the double bonds may be positioned at any location along the backbone. In some embodiments, the alkenyl backbone of the branched alkenyl has two double bonds. In some embodiments, the alkenyl backbone of the branched alkenyl has three double bonds.

One or more alkyl branches are covalently bonded to the backbone of a branched alkenyl. In some embodiments, the branch is an alkyl of at least C₁, at least C₅, or at least C₁₀. In some embodiments, the branch is an alkyl that is no greater than C₁₀, no greater than C₅, or no greater than C₁. In some embodiments, the branch is (C₁ to C₁₅)alkyl or (C₁ to C₁₀)alkyl. In some embodiments, the branch is (C₅ to C₁₀)alkyl. In some embodiments, the branch is a linear alkyl. In some embodiments, the branch is a branched alkyl. In some embodiments, the branch is selected from methyl, ethyl, n-propyl, isopropyl, n-butyl, isobutyl, t-butyl, pentyl, hexyl, heptyl, octyl, nonyl, decyl, undecyl, and dodecyl. In some embodiments, the backbone of the branched alkenyl has a single alkyl branch. In some embodiments, the backbone of the branched alkenyl has two or more alkyl branches. In some embodiments, the alkenyl backbone of the branched alkenyl has two or more double bonds and two or more covalently bonded alkyl branches. In some embodiments, T is citryl. In some embodiments, T is farnesyl.

A^{X} is a second N-terminal cap group or a hydrogen atom (H). In some embodiments A^{X} is H. In embodiments in which A^{X} is not H, A^{X} may be any N-terminal cap group as described relative to T. In embodiments in which A^{X} is not H, A^{X} and T may be the same. For example, in some embodiments, both A^{X} and T are n-tridecyl (C₁₃H₂₇). In other embodiments, both A^{X} and T are both n-hexyl (C₆ H₁₃), sometimes called Ndha. In other embodiments, both A^{X} and T are both n-octyl (C₈H₁₇), sometimes called Ndoa. In embodiments in which A^{X} is not H, A^{X} and T may not be the same.

In some embodiments, Q is hydroxyl. In some embodiments, Q is NH₂.

In Formula I, R¹, R², R³, R⁴, R⁵, and R⁶ are each independently selected from an N-substituted glycine side chain that is covalently bonded to the nitrogen of the peptoid backbone.

Generally, R¹, R², R³, R⁴, R⁵, and R⁶ are each independently selected from an alkyl amine of the general formula R¹⁰NR¹¹R¹²R¹³ (discussed in detail herein); or group having any one of the following general formulas (Nval, Nyalk, Nleun, Ncpen, Nchex, NpenW, NlinW, Nphn, Npea, NphOH, NphX, Nnapn, NnphOR, Nnpyrp, Nnpyrm, Nnpyro, Nnim1, Nnim2, Nchexm, Nnain, Nindn, NpenZ, Narg, sarcosine), or a protonated form thereof:

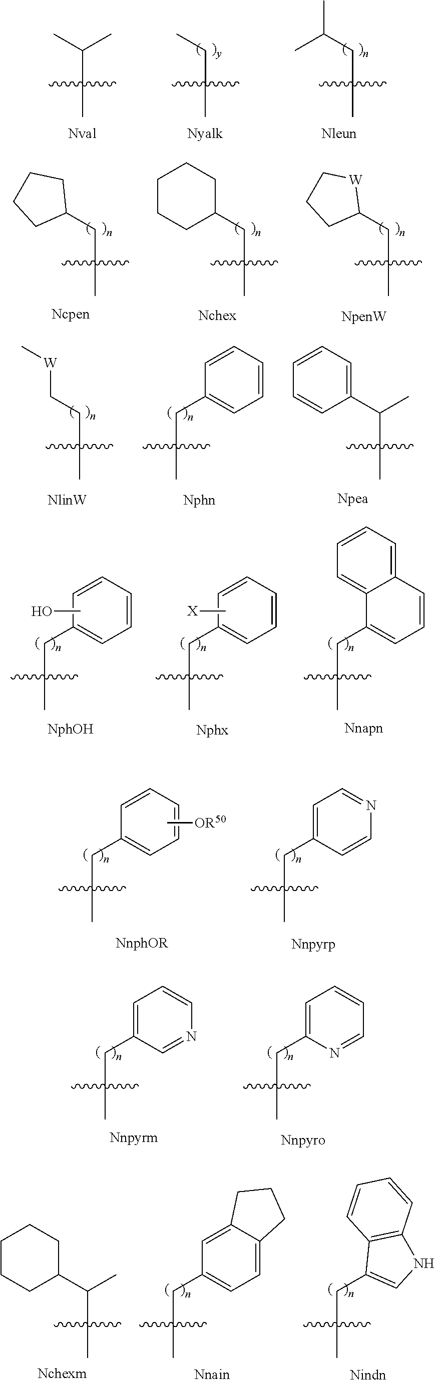

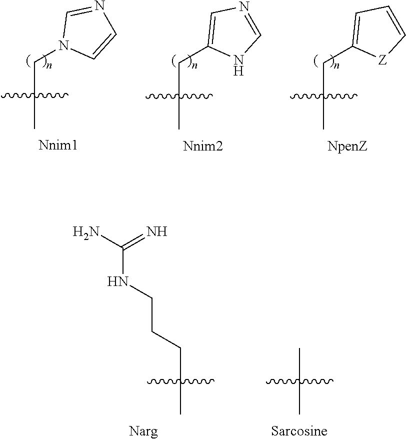

Each n is independently a (C₀, C₁, C₂, or C₃)alkylene (e.g., (C₀ to C₃)alkylene). In some embodiments, where R¹, R², R³, R⁴, R⁵, R⁶, or a combination thereof, are each independently, Nyalk, Nleun, Ncpen, Nchex, NcpenW, NlinW, Nphn, NphOH, NphX, Nnapn, NnphOR, Nnpyrp, Nnpyrm, Nnpyro, Nnim1, Nnim2, Nnain, Nindn, NpenZ, a protonated form thereof, or a combination thereof, each n is independently a (C₀, C₁, C₂, or C₃)alkylene.

In some embodiments, where at least one of R¹, R², R³, R⁴, R⁵, or R⁶, is Npea, the carbon to which the methyl group is attached has R stereochemistry (NxxI). In some embodiments, where at least one of R¹, R², R³, R⁴, R⁵, or R⁶ is Npea, the carbon to which the methyl group is attached has S stereochemistry (Nspe). In embodiments where two or more of R¹, R², R³, R⁴, R⁵, or R⁶ are Npea, each Npea may have the same stereochemistry or different stereochemistry.

In some embodiments, where at least one of R¹, R², R³, R⁴, R⁵, or R⁶ is Nchexm, the carbon to which the methyl group is attached has R stereochemistry (NchexmR). In some embodiments, where at least one of R¹, R², R³, R⁴, R⁵, or R⁶ is Nchexm, the carbon to which the methyl group is attached has S stereochemistry (NchexmS). In embodiments where two or more of R¹, R², R³, R⁴, R⁵, or R⁶ are Nchexm, each Nchexm may have the same stereochemistry or different stereochemistry.

Each y is independently a (C₀, C₁, C₂, C₃, C₄, C₅, or C₆,)alkylene (e.g., (C₀ to C₆)alkylene). In some embodiments, Nyalk is Nhex. In some embodiments, Nyalk is Nnva.

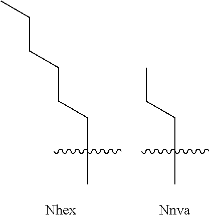

Each R⁵⁰ of NnphOR is independently a (C₁, C₂, or C₃)alkyl. In some embodiments, R⁵⁰ is methyl (C₁), ethyl (C₂), or propyl (C₃). Any substitution pattern of OR⁵⁰ around the NnphOR ring is allowed. For example, the OR⁵⁰ group may be ortho, para, or meta to the alkylene spacer (n) on the ring. According to an embodiment, OR⁵⁰ is para to the alkylene spacer (n) on the ring. In some embodiments, NnphOR is NphOMe.

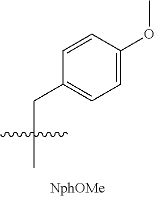 [SMILES: *Cc1ccc(OC)cc1]

Each W in NlinW and NcpenW is independently O, S, or N. In some embodiments, NlinW is Nmea. In some embodiments, NcpenW is Nthf.

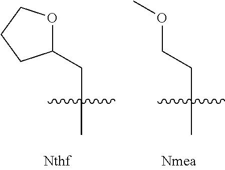

Each X in NphX is independently a halogen, for example fluoro, chloro, bromo, or iodo. Any substitution pattern of X around the NphX ring is allowed. For example, the halogen may be ortho, para, or meta to the alkylene spacer (n) on the ring. In some embodiments, X is para to the alkylene spacer (n) on the ring. In some embodiments, NphX is Npfb. In some embodiments, NphX is Npcb. In some embodiments, NphX is Npib. In some embodiments, NphX is Npbb.

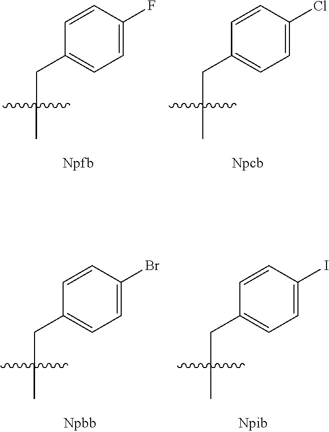 [SMILES: *Cc1ccc(F)cc1 ; *Cc1ccc(Cl)cc1 ; *Cc1ccc(Br)cc1 ; *Cc1ccc(I)cc1]

Any substitution pattern of the hydroxyl around the ring of NphOH is allowed. For example, the hydroxyl may be ortho, para, or meta to the alkylene spacer (n) on the ring. In some embodiments, the hydroxyl is para to the alkylene spacer (n) on the ring. In some embodiments, NphOH is Ntry.

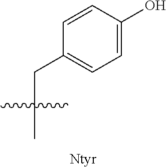 [SMILES: *Cc1ccc(O)cc1]

Each Z of NpenZ is independently NH, S, or O. In some embodiments, NpenZ is Nfur. In some embodiments, NpenZ is Ntma.

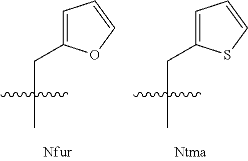 [SMILES: *Cc1ccco1 ; *Cc1cccs1]

In some embodiments, Ncpen is Ncpa. In some embodiments, Nchex is Ncha. In some embodiments, Nchex is Nchm. In some embodiments, Nphn is Nani. In some embodiments, Nphn is Nphe. In some embodiments, Nnapn is Nnap. In some embodiments, Nnapn is Nnapx. In some embodiments, Nnain is Nain. In some embodiments, Nindn is Ntrp. In some embodiments, Nindn is Nhtrp. In some embodiments, Nnpyr is Npyr or a protonated form thereof. In some embodiments, Nnpyrm is Npyrm or a protonated form thereof. In some embodiments, Nnpyro is Npyro or a protonated form thereof. In some embodiments, Nnim1 is Nim1 or a protonated form thereof. In some embodiments Nnim2 is Nim2 or a protonated form thereof.

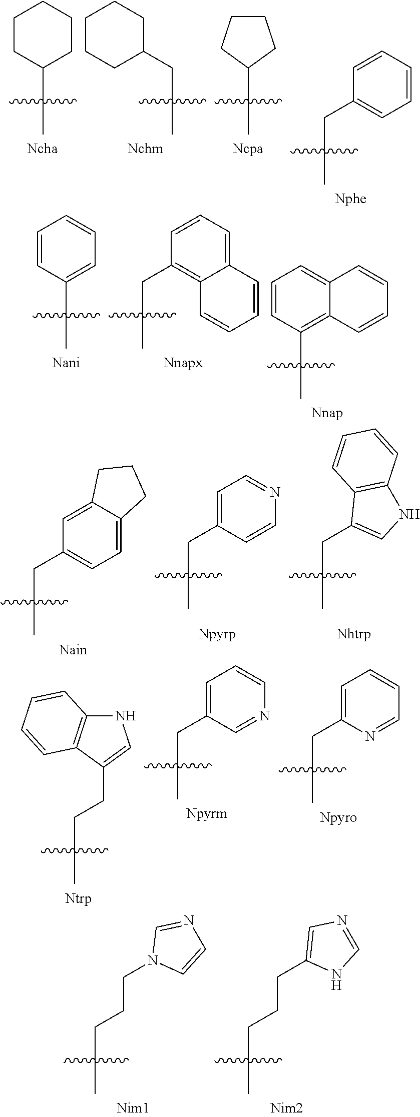

In some embodiments R¹, R², R³, R⁴, R⁵, R⁶ or a combination thereof is an alkyl amine of the general formula R¹⁰NR¹¹R¹²R¹³. R¹⁰ is an alkylene that is covalently bonded to the nitrogen of backbone of the peptoid. In some embodiments, R¹⁰ is of at least C₁ or at least C₃. In some embodiments, R¹⁰ is no greater than C₆ or no greater than C₃. In some embodiments, R¹⁰ is (C₁ to C₆)alkylene. In some embodiments, R¹⁰ is (C₁ to C₃)alkylene. In some embodiments, R¹⁰ is (C₃ to C₆)alkylene. In some embodiments, R¹⁰ is (C₂)alkylene, (C₃)alkylene, or (C₄)alkylene. R¹¹, R¹², and R¹³ are each independently selected from H or (C₁ to C₃)alkyl. In some embodiments, the alkyl chain is methyl, ethyl, or n-propyl. In some embodiments, R¹¹, R¹², and R¹³ are H. In some embodiments, R¹¹ and R¹² are H and R¹³ is (C₁ to C₃)alkyl. In some embodiments, R¹¹ is H, and R¹² and R¹³ are each independently (C₁ to C₃)alkyl. In some embodiments, R¹¹, R¹², and R¹³ are each independently (C₁ to C₃)alkyl. In some embodiments, R¹¹ 0 is H, and R¹² and R¹³ are methyl. In some embodiments, R¹¹, R¹², and R¹³ are methyl. In some embodiments, R¹¹ and R¹² are H and R¹³ is methyl.

In some embodiments, the alkyl amine is Nlys (or a protonated form thereof). In some embodiments, the alkyl amine is Nap (or a protonated form thereof). In some embodiments, the alkyl amine is Nae (or a protonated form thereof). In some embodiments, the alkyl amine is Nlys(me)₃. In some embodiments, the alkyl amine is Nap(me)₃. In some embodiments, the alkyl amine is Nae(me)₃. In some embodiments, the nitrogen of Nlys, Nap, and/or Nae may be protonated, and therefore, possess a formal charge (and therefore are the protonated form of the alkyl amide).

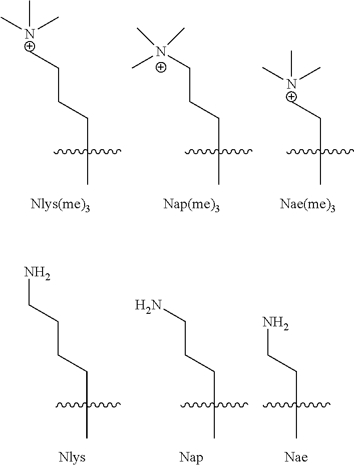

In some embodiments, each one of R¹, R², R⁴, and R⁵ are independently selected from Nval, Nyalk, Nleun, Ncpen, Nchex, NpenW, NlinW, Nphn, Npea, NphOH, NphX, Nnapn, NnphOR, Nnpyrp, Nnpyrm, Nnpyrol, Nnim1, Nnim2, Nnain, Nindn, Npenz, Nchexm, sarcosine, or a protonated form thereof; and each one of R³ and R⁶ are independently selected from Narg (or a protonated form thereof), Nlys (or a protonated form thereof), Nap (or a protonated form thereof), Nae (or a protonated form thereof), Nlys(me)₃, Nap(me)₃, or Nae(me)₃.

In some embodiments, one of R¹, R², R³ R⁴, R⁵, or R⁶ is Nnva; one of R¹, R², R³ R⁴, R⁵, or R⁶ is Npea; one of R², R³ R⁴, R⁵, or R⁶ is Nlys (or a protonated form thereof); one of R¹, R², R³ R⁴, R⁵, or R⁶ is Nfur; one of R¹, R², R³ R⁴, R⁵, or R⁶ is Nphe; and one of R¹, R², R³ R⁴, R⁵, or R⁶ is Nap (or a protonated form thereof). In some embodiments, R¹ is Nnva; R² is Npea; R³ is Nlys (or a protonated form thereof); R⁴ is Nfur; R⁵ is Nphe; and R⁶ is Nap (or a protonated form thereof). In some embodiments, Q is NH₂; A^{X} is H; T is (C₁₃H₂₇)alkyl; R¹ is Nnva; R² is Npea; R³ is Nlys (or a protonated form thereof); R⁴ is Nfur; R⁵ is Nphe; and R⁶ is Nap (or a protonated form thereof). In some embodiments, the peptoid has the structure:

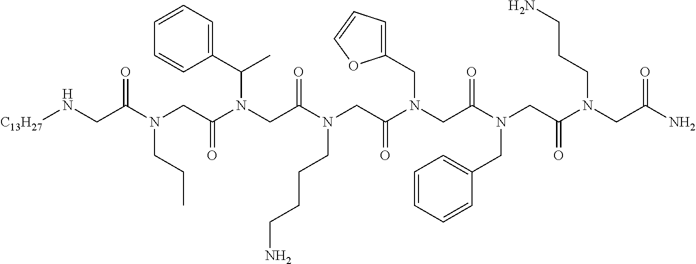

or a protonated form thereof.

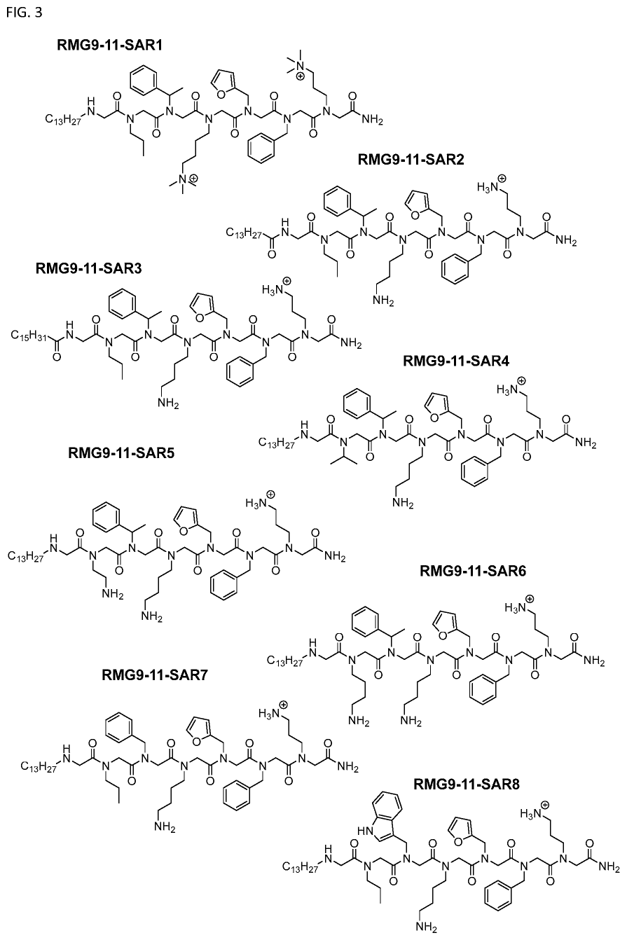

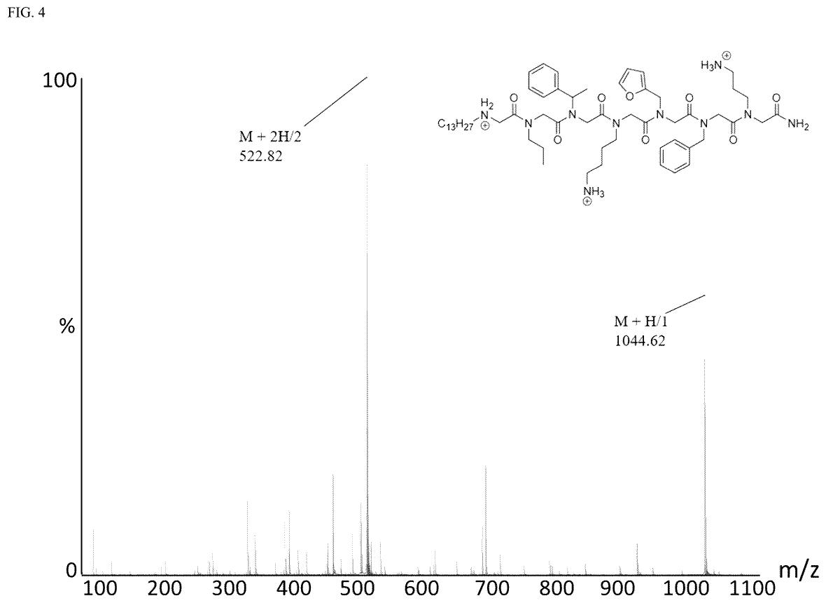

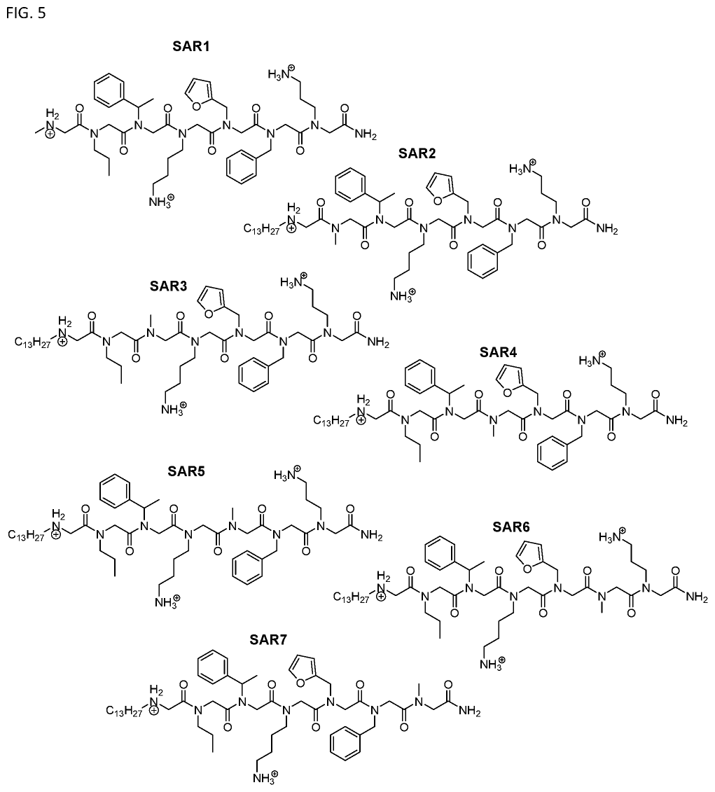

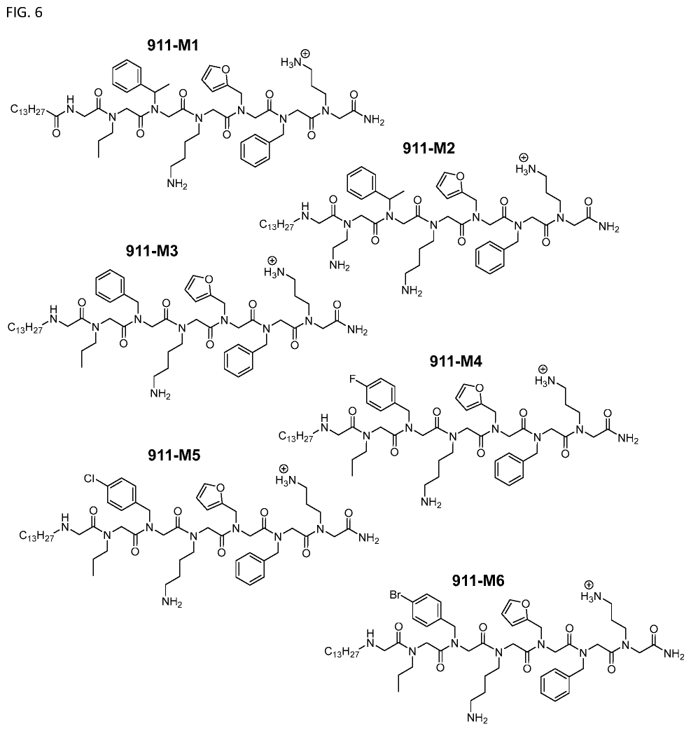

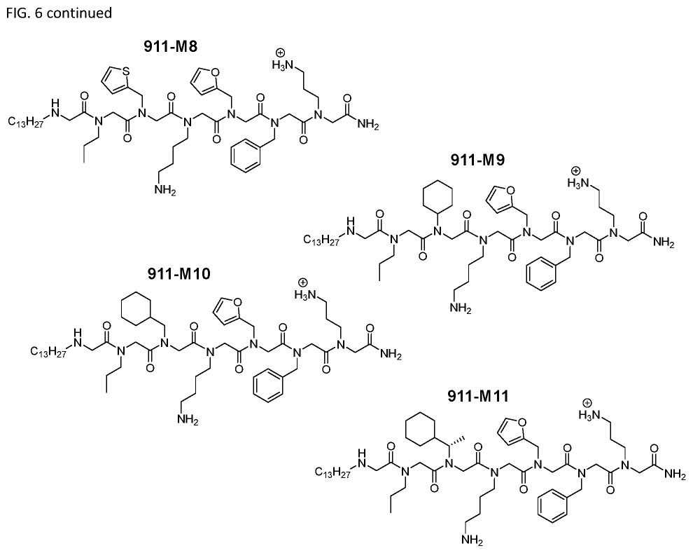

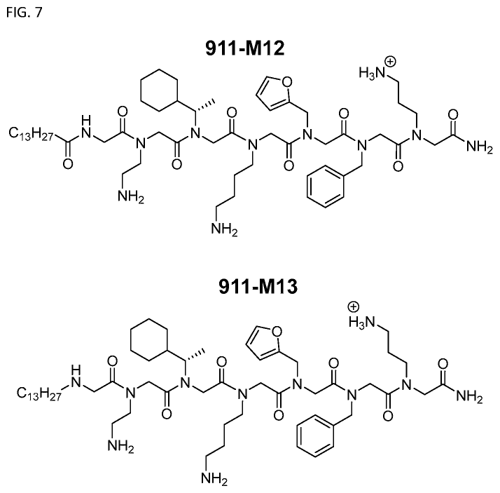

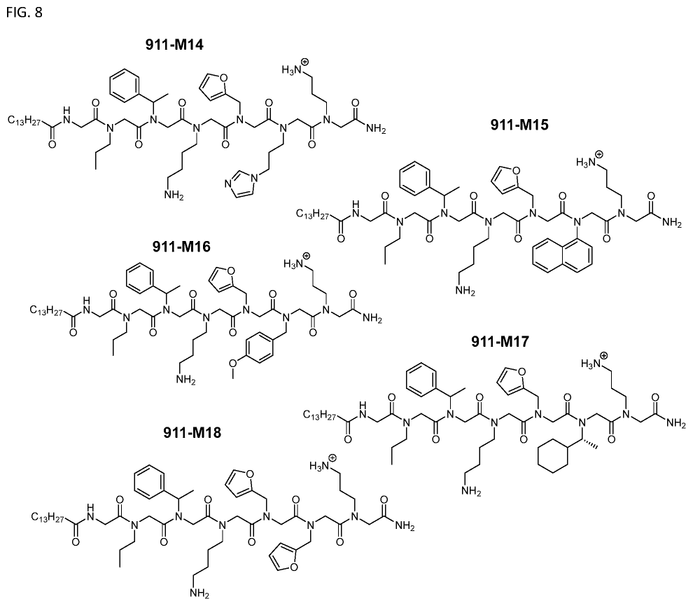

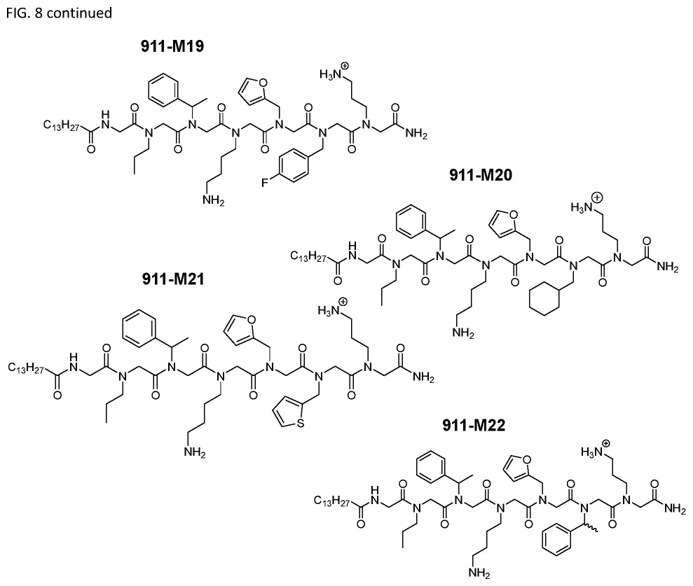

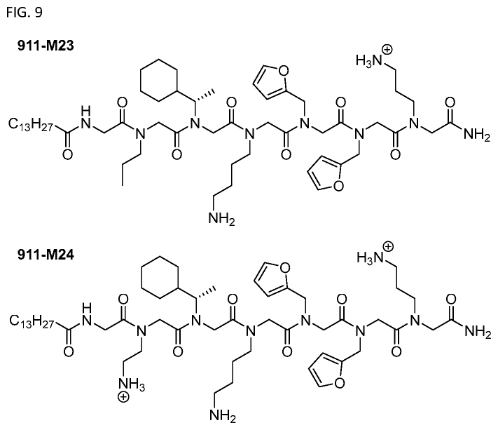

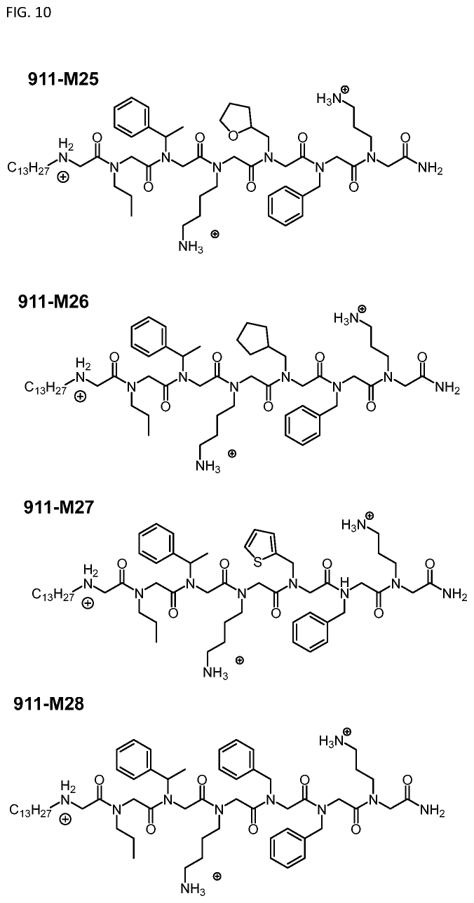

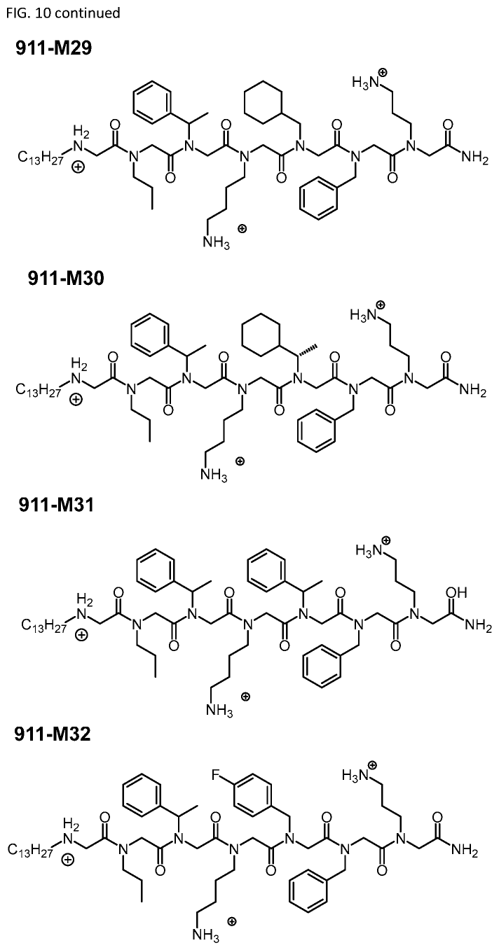

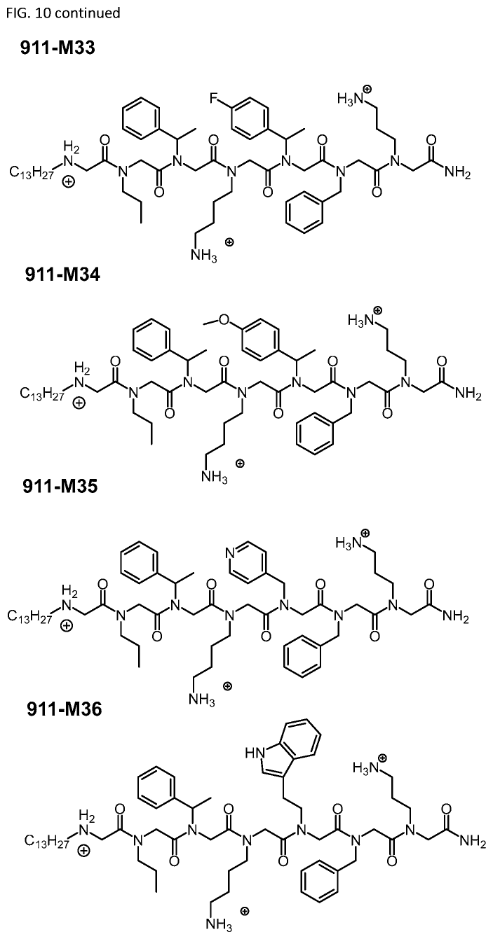

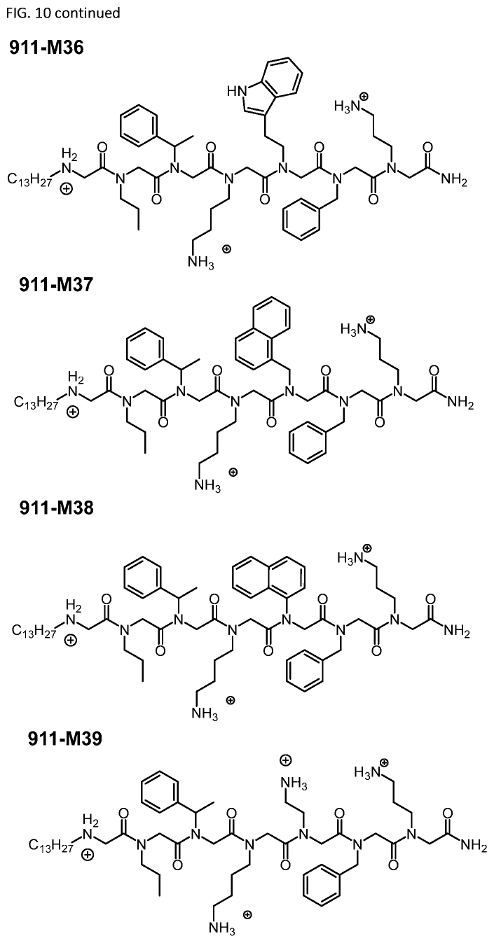

Examples of illustrative peptoids of Formula I are show in FIG. 3 (RMG9-11SAR1; RMG9-11SAR2; RMG9-11SAR3; RMG9-11SAR4; RMG9-11SAR5; RMG9-11SAR6; RMG9-11SAR7; and RMG9-11SAR8, RMG9-11SAR9, RMG9-11SAR 10, RMG9-11SAR11, RMG9-11SAR12, RMG9-11SAR13, RMG9-11SAR14, and RMG9-11SAR15); FIG. 5 (SAR1, SAR2, SAR3, SAR4, SAR5, SAR6, and SAR7); FIG. 6 (9-11-M1, 9-11-M2, 9-11-M3, 9-11-M4, 9-11-M5, 9-11-M6, 9-11-M7, 9-11-M8, 9-11-M9, 9-11-M10, and 9-11-M11); FIG. 7 (9-11-M12 and 9-11-M113); FIG. 8 (9-11-M14, 9-11-M15, 9-11-M16, 9-11-M17, 9-11-M189-11-M19, 9-11-M20, 9-11-M21, and 9-11-M22); FIG. 9 (911-M23and 9-11-M24); and FIG. 10 (9-11-M25, 9-11-M26, 9-11-M27, 9-11-M28, 9-11-M29, 9-11-M30, 9-11-M31, 9-11-M32, 9-11-M33, 9-11-M34, 9-11-M35, 9-11-M36, 9-11-M37, 9-11-M38, and 9-11-M39); or protonated forms thereof.

### Methods of Making Peptoids

Generally, peptoids are synthesized from the N-terminus to the C-terminus using the sub-monomer protocol (Zuckermann et al, J. Am. Chem. Soc. 114, 10646-10647 (1992)). Peptoids may be synthesized on resin with microwave assistance, on resin without microwave assistance, in solution with microwave assistance, or in solution without microwave assistance. Peptoid synthesis generally involves six steps: resin preparation, acylation, amine coupling, cleavage, optional deprotection of protecting groups if protecting groups are present, and termini functionalization. In some embodiments, several of the steps may be combined, for example cleavage from the resin and deprotection of protecting groups may be accomplished at the same time. The peptoids of the present disclosure may be synthesized on resin with microwave assistance.

An exemplary method of peptoid synthesis of the compounds (peptoids) of the general Formula I is as follows. An Fmoc protected Rink Amide resin is prepared by swelling the resin in dimethylformamide (DMF). The Fmoc protecting group is removed by agitating the resin with 10-40% piperidine in DMF for 5-60 min, resulting in a free amine. In some embodiments, Fmoc deprotection is done by agitating the resin with 20% piperidine in DMF for 20 min. The free amine is acylated by mixing the resin, 1-4 M bromoacetic acid, 1-6 M diisopropylcarbodiimide, and DMF to give an amide product with a bromo group on the β carbon. In some embodiments, the amine on the resin is acylated by mixing the resin, 2 M bromoacetic acid, 3.2 M diisopropylcarbodiimide, and DMF. Following acylation, 1-6 M of an amine described by general formula NH₂-R, where R is the desired N-substituted glycine side chain or a protected derivative of the desired N-substituted glycine side chain, is added to the resin. Each R group may include a protecting group, such as a Boc group. A displacement reaction occurs where the bromo group is replaced by the NH₂-R group on the β carbon, thus coupling the amine to the amide formed in the acylation step. In some embodiments, 2 M of an amine of the general formula —NH₂—R is added to the resin to complete the displacement reaction. The acylation and amine coupling steps are repeated until the desired peptoid sequence is reached. Prior to, or after cleavage from the resin, the C-terminus may be functionalized. The peptoid and any Boc protecting groups present on the N-substituted glycine side chains may be cleaved from the resin by incubating a cocktail of trifluoracetic acid, water, and triisopropylsilane for 30 min to 2 hours. In some embodiments the cocktail of trifluoracetic acid, water, and triisopropylsilane is 95:2.5:2.5. In some embodiments, a cocktail of trifluoracetic acid, water, and triisopropylsilane is incubated with the resin for 1 hour. Following cleavage from the resin, the N-terminus may be functionalized. The peptoid may be purified on a reverse-phase HPLC system using a 0-100% water to acetonitrile gradient containing 0.05% trifluoracetic acid. The solvent may be removed in vacuo to yield peptoids as powders.

### Methods of Testing Peptoids

A peptoid library agar diffusion (PLAD) assay may be used to identify peptoids that have antimicrobial activity. The PLAD assay may be accomplished using methods as described in Fischer et al., ACS Comb. Sci. 18, 287-291 (2016); Corson et al., ACS Med. Chem. Lett. 7, 1139-1144 (2016); Turkett et al., ACS Comb. Sci. 18, 287-291 (2016), which is incorporated by reference herein. The PLAD assay uses a PLAD chemical linker. Please see Example 1 for details on the PLAD linker construction and synthesis. The PLAD chemical linker displays two identical strands, α and β, of a peptoid of interest where each strand can be released in response to different chemical stimuli (Fischer et al., ACS Comb. Sci. 18, 287-291 (2016)). Beads containing the PLAD linked peptoids are embedded in an agar medium where the agar medium is inoculated with the microbe of interest. The β-strand peptoid is released by a reducing reagent. The reducing agent cleaves a disulfide within the PLAD linker, allowing the β-strand peptoid to interact with microbes around the bead. If the released peptoid has antimicrobial activity, a zone of inhibited growth will be present. The α-strand peptoid remains attached to the bead during the screening process. The α-strand peptoid is cleaved from a bead that displays a zone of inhibited growth. The chemical cleavage of the α-strand peptoid may be accomplished at a C-terminal methionine of the PLAD linker using cyanogen bromide. Mass spectrometry sequencing is used to determine the structure of the α-strand peptoid corresponding to the β-strand peptoid that displayed a zone of inhibited growth. The PLAD assay may be adapted to screen a peptoid of interest against different types of microbes of interest through slight modifications of the assay parameters. Examples of microbes that may be employed in a PLAD assay include fungal pathogens and bacterial pathogens. Examples of fungal pathogens include, but are not limited to, C. albicans, C. tropicalis, C. stellatoidea, C. glabrata, C. krusei, C. parapsilosis, C. guilhermondii, C. viswanathii, C. lusitaniae, C. auris, R. mucilaginosa, R. minuta, or R. glutinis, C. neoformans, and Cryptococcus gattii. Examples of bacterial pathogens include, but are not limited to, Enterococcus faecium, Staphylococcus aureus, Enterococcus faecalis, Pseudomonas aeruginosa, Enterobacter, Klebsiella pneumoniae, Escherichia coli, Acinetobacter baumannii, and Mycobacterium smegmatis.

An inverted Peptoid Library Agar Diffusion (iPLAD) assay may be used to assess the antimicrobial activity of a peptoid against a biofilm produced from a microbe of interest. Example 1 gives an exemplary method that can be used to complete the iPLAD assay. Briefly, the iPLAD assay can be understood as having four stages. In the first stage, a culture of a fungal pathogen is allowed to grow and produce a biofilm on the surface of a Petri dish. In the second stage, excess media is removed from the Petri dish and the biofilm is washed. A PLAD linked peptoid (or library) bead and reducing reagent, both suspended in a minimal amount soft liquified agar, are added to the biofilm. In the third stage, the PLAD linked peptoid is incubated overnight with the biofilm. The reducing reagent releases the β-strand peptoid from library beads to interact with the biofilm. After incubation, an imaging reagent is added on top of the soft agar and allowed to diffuse into the biofilm, revealing “hit” peptoids that have killed fungal cells within the biofilm. In stage four, the hit peptoids are isolated and the structure of the α-strand of the active peptoid is deconvoluted by tandem mass spectrometry. Examples of microbes that may be employed in a PLAD assay include fungal pathogens and bacterial pathogens. Examples of fungal pathogens include, but are not limited to, C. albicans, C. tropicalis, C. stellatoidea, C. glabrata, C. krusei, C. parapsilosis, C. guilhermondii, C. viswanathii, C. lusitaniae, C. auris, R. mucilaginosa, R. minuta, or R. glutinis, C. neoformans, and Cryptococcus gattii. Examples of bacterial pathogens include, but are not limited to, Enterococcus faecium, Staphylococcus aureus, Enterococcus faecalis, Pseudomonas aeruginosa, Enterobacter, Klebsiella pneumoniae, Escherichia coli, Acinetobacter baumannii, and Mycobacterium smegmatis.

A minimum inhibitory concentration (MIC) assay may be used to determine the activity of a peptoid against a microbe of interest. Example 1 gives an exemplary method that can be used to complete a MIC assay. A MIC assay may be conducted via the guidelines sort forth in the Clinical and Laboratory Standards Institute (CLSI). In an example MIC assay, a peptoid of interest is diluted into a solution containing the microbe of interest. The peptoid-microbe solution is incubated at a set temperature, for example 37° C., for a set amount of time. The growth, or lack of growth, of the microbe after incubation is determined by manual observation or with the aid of a cell viability dye and fluorescent measurements. A MIC assay may be conducted with technical and biological replicates. A compound known to kill the microbe of interest may be included as a positive control. The MIC is generally defined as the lowest concentration of the peptoid of interest that prevents microbe growth. Examples of microbes that may be employed in a MIC assay include fungal pathogens and bacterial pathogens. Examples of fungal pathogens include, but are not limited to, C. albicans, C. tropicalis, C. stellatoidea, C. glabrata, C. krusei, C. parapsilosis, C. guilliermondii, C. viswanathii, C. lusitaniae, C. auris, R. mucilaginosa, R. minuta, or R. glutinis, C. neoformans, and Cryptococcus gattii. Examples of bacterial pathogens include, but are not limited to, Enterococcus faecium, Staphylococcus aureus, Enterococcus faecalis, Pseudomonas aeruginosa, Enterobacter, Klebsiella pneumoniae, Escherichia coli, Acinetobacter baumannii, and Mycobacterium smegmatis.

In some embodiments, a microbe is unable to develop resistance to the peptoids of the present disclosure. Antimicrobial compounds that prevent the emergence of resistant variants are highly desirable therapeutic agents, as antimicrobial strategies typically become obsolete when a target microbe develops resistance. A peptoid of the present disclosure can have the activity of reducing the development of resistance in a target microbe. A serial passage gain of resistance assay may be used to evaluate the ability of a microbe of interest to gain resistance to a peptoid of interest, and thereby determine whether the peptoid has the activity of reducing the development of resistance in a target microbe. An exemplary serial passage gain of resistance assay is described in Example 1. Examples of microbes that may be employed in a serial passage gain of resistance assay include fungal pathogens and bacterial pathogens. Examples of fungal pathogens include, but are not limited to, C. albicans, C. tropicalis, C. stellatoidea, C. glabrata, C. krusei, C. parapsilosis, C. guilliermondii, C. viswanathii, C. lusitaniae, C. auris, R. mucilaginosa, R. minuta, or R. glutinis, C. neoformans, and Cryptococcus gattii. Examples of bacterial pathogens include, but are not limited to, Enterococcus faecium, Staphylococcus aureus, Enterococcus faecalis, Pseudomonas aeruginosa, Enterobacter, Klebsiella pneumoniae, coli, Acinetobacter baumannii, and Mycobacterium smegmatis.

A single passage gain of resistance assay may be used to evaluate the ability of a microbe of interest to gain resistance to a peptoid of interest, and thereby determine whether the peptoid has the activity of reducing the development of resistance in a target microbe. An exemplary single passage gain of resistance assay is described in Example 1. Examples of microbes that may be employed in a MIC assay include fungal pathogens and bacterial pathogens. Examples of fungal pathogens include, but are not limited to, C. albicans, C. tropicalis, C. stellatoidea, C. glabrata, C. krusei, C. parapsilosis, C. guilliermondii, C. viswanathii, C. lusitaniae, C. auris, R. mucilaginosa, R. minuta, or R. glutinis, C. neoformans, and Cryptococcus gattii. Examples of bacterial pathogens include, but are not limited to, Enterococcus faecium, Staphylococcus aureus, Enterococcus faecalis, Pseudomonas aeruginosa, Enterobacter, Klebsiella pneumoniae, Escherichia coli, Acinetobacter baumannii, and Mycobacterium smegmatis.

The quotient of the MIC obtained prior to the gain of resistance assay and the MIC obtained during the gain of resistance assay may be used to quantify the ability of a microbe to develop resistance to a peptoid of interest. Examples of a gain or resistance assay include, but are not limited to, a serial passage gain of resistance assay and a single passage gain of resistance assay. Although no lower limit is desired, in practice the MIC of the gain of resistance assay may be no greater than 200%, no greater than 150%, no greater than 125%, no greater than 105% or no greater than 102% of the MIC obtained prior to the gain of resistance assay. In some embodiments, the MIC of the gain of resistance assay may be no less than 150%, no less than 125%, no less than 105%, or no less than 102% of the of the MIC obtained prior to the gain of resistance assay. In some embodiments, the MIC of the gain of resistance assay may be 102% to 200%, 102% to 150%, 102% to 125%, or 102% to 105% of the of the MIC obtained prior to the gain or resistance assay. In some embodiments, the MIC of the gain of resistance assay may be 105% to 125%, 105% to 150%, or 105% to 200% of the of the MIC obtained prior to the gain of resistance assay. In some embodiments, the MIC of the gain or resistance assay may be 125% to 150% or 125% to 200% of the of the MIC obtained prior to the gain or resistance assay. In some embodiments, the MIC of the gain of resistance assay may be 50% to 200% of the of the MIC obtained prior to the gain or resistance assay. In one embodiment, a microbe that is unable to develop resistance to a peptoid of the present disclosure is a Candida spp., such as, for example, C. albicans, C. tropicalis, C. stellatoidea, C. glabrata, C. krusei, C. parapsilosis, C. guilhermondii, C. viswanathii, C. auris, or C. lusitaniae.

Peptoids of interest may be evaluated against different strains of pathogenic microbes. For example, a MIC assay may be used to assess the activity of a peptoid of interest against C. albicans, a biofilm produced by C. albicans, C. albicans M1 (a reference strain), C. albicans M2, C. albicans M3, C. albicans M4, C. albicans M5, C. albicans M6, C. albicans M7, and C. albicans ATCC 64124. It may be beneficial to evaluate the activity of peptoids of interests against strains known to be resistant to specific therapeutic treatments. For example, C. albicans M2, C. albicans M3, and C. albicans M5 are resistant to fluconazole treatment. Additionally, ATCC 64124 is a multidrug resistant C. albicans strain that resistant to amphotericin B, fluconazole, caspofungin, and flucytosine. Peptoids displaying activity against drug resistant microbe strains may be advantageous for treating subjects infected with the drug resistant microbe strains. Peptoids displaying activity against drug resistant microbe strains may be advantageous for treating subjects with microbe infections as microbes are less likely to develop resistance to these peptoids.

Peptoids of interest may be evaluated via in vivo experiments to assess their use as therapeutic treatments. In vivo organism models may include, but are not limited to, plant, mouse, rat, cat, pig, cow, monkey, and human.

### Compositions and Methods of Use

In another aspect, this disclosure describes compositions (e.g., pharmaceutical compositions and fungicidal compositions) that include at least one of the peptoids described herein, or a salt thereof, as an active ingredient. Within the context of this disclosure, recitation of peptoid is understood to include the peptoid as a free base and/or a salt, such as a pharmaceutically acceptable salt of the peptoid. The term “free base” refers to the conjugate base (unprotonated) of an amine or amines. A pharmaceutically acceptable salt of a peptoid refers to an ionized or ionizable drug substance that has been neutralized through one or more ionic bonds to an appropriate counterion (e.g., and anion or cation). Any peptoid as described herein may be the active ingredient in any composition described herein.

In embodiments, the composition is a pharmaceutical composition. The pharmaceutical composition may be formulated with a pharmaceutically acceptable carrier. As used herein, “carrier” includes any solvent, dispersion medium, vehicle, coating, diluent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. As used herein, “pharmaceutically acceptable” refers to a material that is not biologically or otherwise undesirable, e.g., the material may be administered to an individual along with the peptoid or pharmaceutically acceptable salt thereof, without causing any undesirable biological effects or interacting in a deleterious manner with any of the other components of the pharmaceutical composition in which it is contained.

At least one of the peptoids is formulated in a pharmaceutical composition and then, in accordance with methods of the disclosure, administered to a subject, such as a mammal (e.g., human, companion animal, or domesticated animal) in a variety of forms adapted to the chosen route of administration. The formulations include those suitable for oral, rectal, vaginal, topical, nasal, ophthalmic or parenteral (including subcutaneous, intramuscular, intraperitoneal, and intravenous) administration. The formulations may be conveniently presented in a form suitable for delivery by a given administration route, and may be prepared by methods well known in the art of pharmacy.

In some embodiments, a peptoid is formulated in combination with one or more additional active agents, such as an antifungal compound. Essentially any known therapeutic agent may be included as an additional active agent. The action of the additional active agent in the combination therapy may be cumulative to the peptoid or it may be complementary, for example, to manage side effects or other aspects of the subject's medical condition. In some embodiments, the combination therapy includes an azole, a polyene, fluorocytosine, amphotericin B, fluconazole, and/or an echinocandin.

In some embodiments, the composition is a fungicidal composition. The fungicidal composition includes as an active agent a peptoid described herein, or salt thereof, and a fungicidal acceptable carrier. As used herein, “fungicidal acceptable” refers to a material that is not biologically or otherwise undesirable, e.g., the material may be administered to a plant along with the peptoid or fungicidal acceptable salt thereof, without causing any undesirable biological effects or interacting in a deleterious manner with any of the other components of the fungicidal composition in which it is contained. At least one peptoid of the present disclosure is formulated in a fungicidal composition and then, in accordance with methods of the disclosure, administered to a plant, the seeds of a plant, or the soil in which the plant grows. The formulations include those suitable for treating the soil in which the plant grows or the plant directly. Types of formulations may include, baits, gels, dusts, water dispersible granules, dry powders, soluble powders, dry granules, pellets, emulsions, solutions, suspensions, impregnated products, fertilizer combinations, or aerosols.

The fungicidal acceptable carrier may include an excipient. An excipient may include, for example, a diluent, a solvent, or an adjuvant. Adjuvants may include compatibility agents, activating agents, buffers, anti-foaming agents, spray colorants, drift control agents, water conditioners, surfactants, or combinations thereof.

In some embodiments, a peptoid is formulated in combination with one or more additional active agents, such as a fungicidal compound. Any known plant treatment agent may be included as an additional active agent. The action of the additional active agent in the combination therapy may be cumulative to the peptoid or it may be complementary. In some embodiments, the combination therapy includes one or more fungicides, such as but not limited to, azoxystrobin, benomyl, propiconazole, tricyclazole, carbendazim, metalaxyl, difenocanazole, hexaconazle, acibenzolar, polyoxin D salt, fluoxastrobin, carbonic acid, mono and dipotassium salts of phosphorus acid, cymoxanil, chlorothalonil, tebuconazole, copper chloride, copper hydroxide, mineral oil, pyraclostrobin, copper sulfate, cymoxanil, mancozeb, boscalid, trifluxystrobin, dimethomorph, sodium percarbonate, thiophanate-methyl, cuprammonium acetate, sulfur, tebuconazole, fosetyl-Al, myclobutanil, cyazofamid, fenamidone, myclobutanil, kresoxim-methyl, or metrafenone.

In embodiments, the peptoids of the present disclosure, or pharmaceutical compositions containing the same, may find utility in the treatment, control or prevention of fungal or bacterial infection and disease in subjects such as vertebrates (e.g., human, a companion animal, or a domesticated animal). Therapeutic treatment may be initiated prior to the development of symptoms of an infection; prior to diagnosis; after diagnosis; or after the development of symptoms of infection with a fungus, bacterium, or both. The active agent (e.g., a peptoid of the present disclosure or a composition containing the same) is administered to a subject in an amount effective to produce the desired effect. As such, an effective amount of a peptoid may be administered to a subject to treat a fungal infection, a bacterial infection, or both. An effective amount is an amount suitable to reduce symptoms; prevent the expansion of symptoms; kill one or more microbes of the infection; or clear the infection from the subject.

The formulations may be administered as a single dose or in multiple doses. Useful dosages of the active agents may be determined by comparing their in vitro activity and the in vivo activity in animal models. For some infectious diseases, a surrogate of the pathogenic bacteria or fungus may be used to gather initial data. For example, M smegmatis may be used as a surrogate for M. tuberculosis. Methods for extrapolation of effective dosages in mice, and other animals, to humans are known in the art.

A peptoid, or a pharmaceutical composition containing the same, may be used to treat or prevent fungal infections, bacterial infections, biofilms produced from fungal infections, biofilms produced from bacterial infections, biofilms produced from fungal and bacterial infection, or combinations thereof. Exemplary fungal infections include, but are not limited to, an infection with a Candida spp. including, for example, C. albicans, C. tropicalis, C. stellatoidea, C. glabrata, C. krusei, C. parapsilosis, C. guilliermondii, C. viswanathii, C. auris, C. lusitaniae, or a combination thereof; an infection with Rhodotorula spp, including, for example, R. mucilaginosa, R. minuta, R. glutinis, or a combination thereof; and/or an infection with Cryptococcus spp. for example, C. neoformans, Cryptococcus gattii, or a combination thereof. Example bacterial infections include infection with a gram-negative microbe, a gram-positive microbe, or a mycobacterium. Examples of gram-negative bacterium include Pseudomonas aeruginosa, Enterobacter, Klebsiella pneumoniae, Escherichia coli, or Acinetobacter baumannii, or a combination thereof. Examples of gram-positive bacterium include Enterococcus faecium, Staphylococcus aureus, Enterococcus faecalis, or a combination thereof. An example of a Mycobacterium is Mycobacterium tuberculosis.

A peptoid of the present disclosure, or pharmaceutical composition containing the same, may also be administered prophylactically, to prevent or delay the development of infection with a fungus, bacterium, or both. Treatment that is prophylactic, for instance, may be initiated before an at risk subject manifests symptoms of infection with a fungus or a bacterium. As used herein, the term “at risk” refers to a subject that may or may not actually possess the described risk. Thus, for example, a subject “at risk” of an infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe. An example of a subject that is at particular risk of developing infection with a fungus or a bacterium is an immunocompromised person. Treatment may be performed before, during, or after the diagnosis or development of symptoms of infection. Treatment initiated after the development of symptoms may result in decreasing the severity of the symptoms of one of the conditions, or completely removing the symptoms. A peptoid of the present disclosure, or pharmaceutical composition containing the same, may be introduced into the subject (e.g., a vertebrate, such as a mammal) at any stage of fungal or bacterial infection.

Administration of a peptoid of the present disclosure, or a pharmaceutical composition containing the same, may occur before, during, and/or after other treatments. Such combination therapy may involve the administration of a peptoid, or pharmaceutical composition containing the same, during and/or after the use of other antifungal or antibacterial agents. The administration of a peptoid, or pharmaceutical composition containing the same, may be separated in time from the administration of other antifungal agents by hours, days, or even weeks.

It should be understood that administration of the peptoid or composition containing the same, to a subject such as a human patient, may be effective to reduce or eliminate fungal or bacterial infection or the symptoms associated therewith; to halt or slow the progression of infection or symptoms within a subject; and/or to control, limit or prevent the spread of infection within a population, or movement of infection to another population.

The peptoids of the present disclosure, or compositions containing the same, may find utility in the treatment, control or prevention of fungal or bacterial infection and disease not only in humans, but also in animals. Peptoids of the present disclosure, or pharmaceutical composition containing the same, may be administered to companion animals, domesticated animals such as farm animals, animals used for research, or animals in the wild. Companion animals include, but are not limited to, dogs, cats, hamsters, gerbils and guinea pigs. Domesticated animals include, but are not limited to, cattle, horses, pigs, fowl, goats, and llamas. Research animals include, but are not limited to, mice, rats, dogs, apes, and monkeys. Administration may be, for example, part of a small-or large-scale public health infection control program. The peptoid, or composition containing the same, may, for example, be added to animal feed as a prophylactic measure for reducing, controlling or eliminating fungal infection or bacterial infection in a wild or domestic animal population. The peptoid, or composition containing the same, may, for example, be administered as part of routine or specialized veterinary treatment of a companion or domesticated animal or animal population.

It should be understood that administration of the peptoid of composition containing the same, to an animal may be effective to reduce or eliminate fungal or bacterial infection or the symptoms associated therewith; to halt or slow the progression of infection or symptoms within a subject; and/or to control, limit or prevent the spread of infection within a population, or movement of infection to another population.

In some embodiments, the peptoids of the present disclosure, or fungicidal compositions containing the same, may find utility in the treatment, control or prevention of fungal infections of plants. As such, in some embodiments, this disclosure describes a method that includes administering to a plant a fungicidal composition that includes an effective amount of a peptoid, or fungicidal composition containing the same.

Exemplary fungal infections include, but are not limited to, an infection with Rhizoctonia solani, Sphaeropsis, Phoma clematidina, Peronosporaceae, Plasmodiophora brassicae, Diplocarpon rosae, Pythium, Phytophthora, Colletotrichum, Gloeosporium, Sclerotinia homoeocarpa, Physoderma, Laetisaria fuciformis, Serpula lacrymans, Synchytrium endobioticum, Ascomycota, Phytophthora infestans, Alternaria solani, Fusarium oxysporum, Verticillium longisporum, Taphrina deformans, Botrytis, Guignardia bidwellii, Venturia inaequalis, Pleurotus ostreatus, Sclerotium rolfsii, Fibroporia vaillantii, Phoma terrestris, Monilinia oxycocci, Ustilago maydis, Phytophthora, Coniophora puteana, Poria vaillantii, Chaetomium, Ceratocystis, Pyrenophora tritici-repentisa, or a combination thereof. This disclosure provides a therapeutic method of treating a plant that has, or is at risk of developing, a fungal infection by administering a peptoid of the present disclosure, or fungicidal composition containing the same, to the subject. Therapeutic treatment is initiated before diagnosis, before the development of symptoms of an infection with a fungus, after diagnosis, or after the development of symptoms of infection with a fungus.

A peptoid of the present disclosure, or fungicidal composition containing the same, may also be administered prophylactically, to prevent or delay the development of infection with a fungus. Treatment that is prophylactic, for instance, may be initiated before a plant manifests symptoms of infection with a fungus. A peptoid of the present disclosure, or fungicidal composition containing the same, may be introduced into the plant at any stage of fungal infection.

Administration of a peptoid of the present disclosure, or fungicidal composition containing the same, to plants may be a part of a small-or large-scale plant health infection control program. The peptoid, or fungicidal composition containing the same, may, for example, be added to fertilizer as a prophylactic measure for reducing, controlling or eliminating fungal infection in a crop population. It should be understood that administration of the peptoid, or fungicidal composition containing the same, may be effective to reduce or eliminate fungal infection or the symptoms associated therewith; to halt or slow the progression of infection or symptoms within a subject; and/or to control, limit or prevent the spread of infection within a population, or movement of infection to another population.

In embodiments, the peptoids of the present disclosure, or compositions containing the same, may find utility as a health or dietary supplement. As such, a peptoid of the present disclosure, or composition containing the same, may be packaged as a nutritional, health or dietary supplement (for example, in pill or capsule form). Additionally, a peptoid, or composition containing the same, may be added to a food product to yield what is commonly referred to as a “nutraceutical” food or “functional” food. Foods to which a peptoid, or composition containing the same, may be added include, without limitation, animal feed; cereals; milk products such as yogurts, cottage cheeses; oils including hydrogenated or partially hydrogenated oils; soups; and beverages. Peptoids having one or more lipophilic or hydrophobic substitutions are preferably incorporated into oily or fatty food products, to facilitate solubilization.

The invention is defined in the claims. However, below there is provided a non-exhaustive listing of non-limiting exemplary aspects. Any one or more of the features of these aspects may be combined with any one or more features of another example, embodiment, or aspect described herein.

### EXEMPLARY ASPECTS

Aspect 1. Aspect 1 is a compound of the general formula,

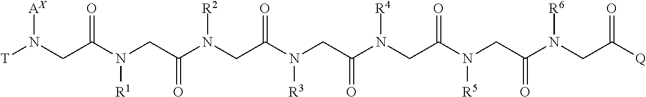

a protonated form thereof, or a pharmaceutically acceptable salt thereof,
wherein:
AX is H or a linear or branched (C6 to C20)alkyl or a linear or branched (C6 to C20)alkenyl, wherein the alkyl or the alkenyl optionally includes a carbonyl group;
T is a linear or branched (C6 to C20)alkyl or a linear or branched (C6 to C20)alkenyl, wherein the alkyl or the alkenyl optionally includes a carbonyl group;
Q is a hydroxyl or NH2; and
R1, R2, R3, R4, R5, and R6 are each independently

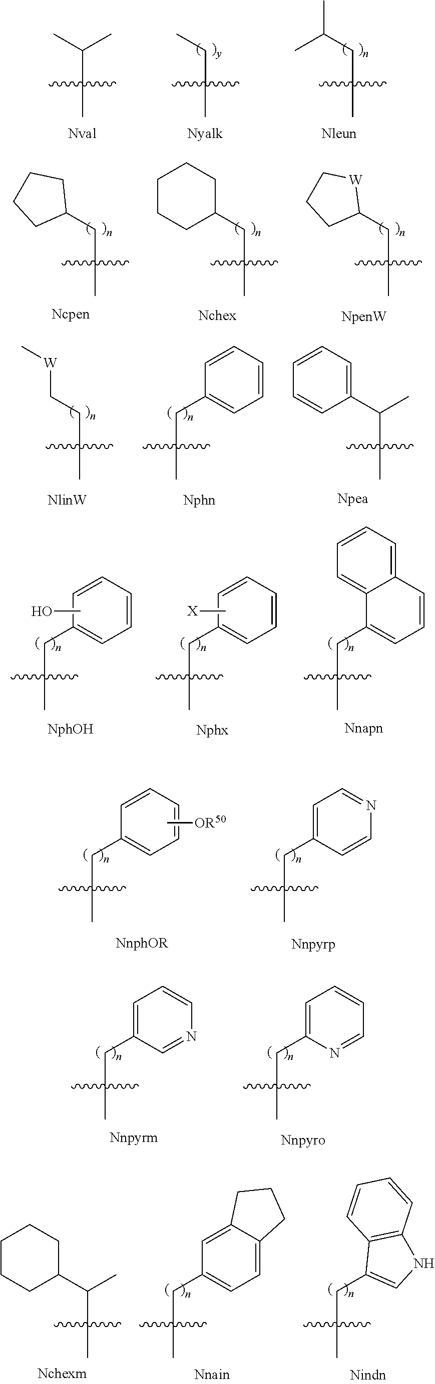

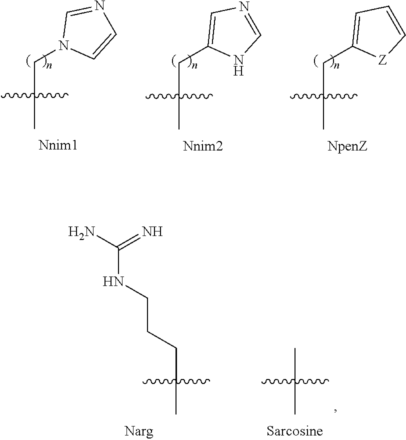

or a protonated form thereof, or an alkyl amine of the general formula R10NR11R12R13,
wherein:
each n is independently (C₀ to C₃)alkylene;
each y is independently (C₀ to C⁶)alkylene;
for each R¹, R², R³, R⁴, R⁵, and/or R⁶ that is NcpenW, each W is independently N, S, or O;
for each R¹, R², R³, R⁴, R⁵, and/or R⁶ that is NlinW, each W is independently N, S, or O;
for each R¹, R², R³, R⁴, R⁵, and/or R⁶ that is NphX, each X is independently F, Cl, Br, or I;
for each R¹, R², R³, R⁴, R⁵, and/or R⁶ that is NapenZ, each Z is independently S, or O;
for each R¹, R², R³, R⁴, R⁵, and/or R⁶ that is R10NR11R12R13, each R10 is independently a linear (C₁ to C₆)alkylene;
for each R¹, R², R³, R⁴, R⁵, and/or R⁶ that is NnphOR, each R⁵⁰ is independently (C₁ to C₃)alkyl; and
for each R¹, R², R³, R⁴, R⁵, and/or R⁶ that is R¹⁰NR¹¹R¹²R¹³, each R¹¹, each R¹², and each R¹³ are independently H or (C₁ to C₆)alkyl.

Aspect 2. Aspect 2 is the compound of Aspect 1, wherein R¹, R², R³, R⁴, R⁵, and R⁶ are each independently

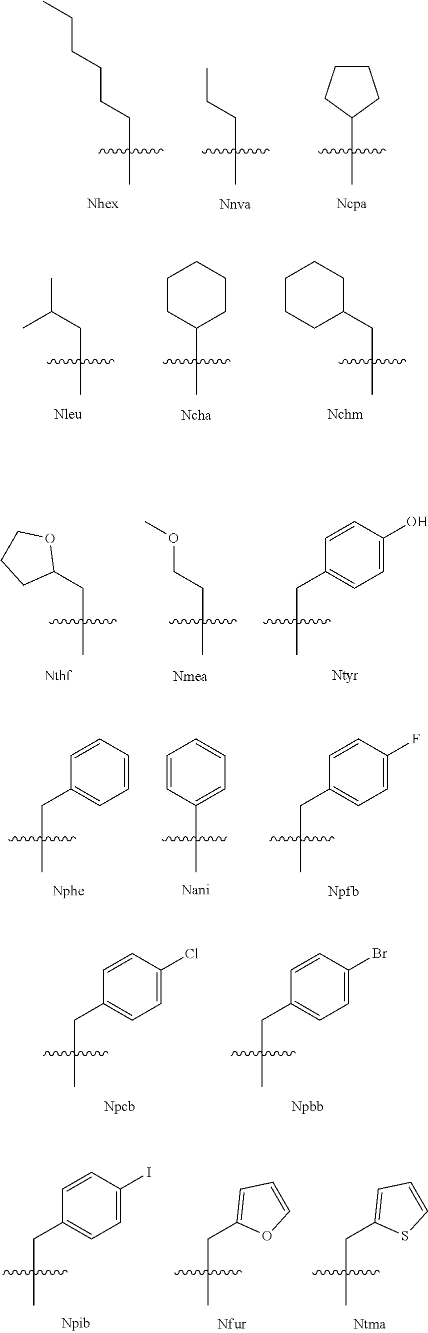

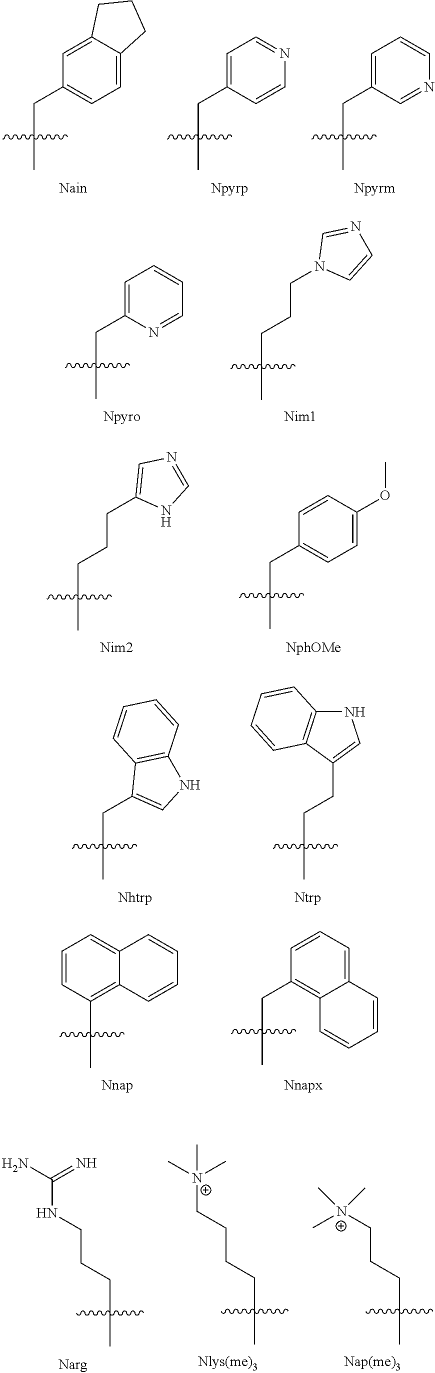

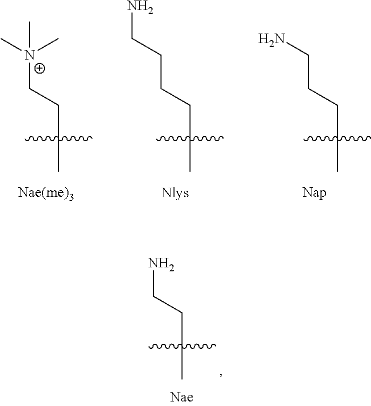

or a protonated form thereof.

Aspect 3. Aspect 3 is compound of Aspects 1-2, wherein T is linear (C₁₃)alkyl; linear (C₁₅)alkyl; citryl; farnesyl; or (CO)R⁵⁰ wherein R⁵⁰ is linear (C₁₃)alkyl or linear (C₁₅)alkyl.

Aspect 4. Aspect 4 is the compound of Aspects 1-3, wherein R¹⁰ is a linear (C₂-C₄)alkylene.

Aspect 5. Aspect 5 is the compound of Aspects 1-4, wherein R¹¹, R¹², and R¹³ are methyl.

Aspect 7. Aspect 6 is the compound of Aspects 1-5 wherein any one of R¹, R², R³, R⁴, R⁵, and R⁶ is Nnva; any one of R¹, R², R³, R⁴, R⁵, and is Npea; any one of R¹, R², R³, R⁴, R⁵, and R⁶ is Nlys; any one of R¹, R², R³, R⁴, R⁵, and R⁶ is Nfur; any one of R¹, R², R³, R⁴, R⁵, and R⁶ is Nphe; and any one of R¹, R², R³, R⁴, R⁵, and R⁶ is Nap. In some such aspects, T is a linear (C₁₃)alkyl or (CO)R⁵⁰ wherein R⁵⁰ is linear (C₁₃)alkyl or linear (C₁₅)alkyl; and Q is —NH₂.

Aspect 7. Aspect 5 is the compound of Aspects 1-5 having the structure

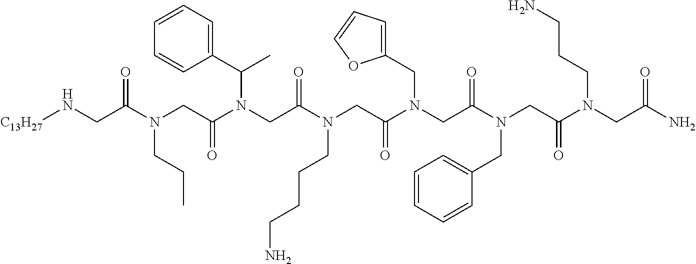

or a protonated form thereof.

Aspect 8. Aspect 8 is a composition comprising the compound of any one of Aspects 1through 7 and a pharmaceutically acceptable carrier.

Aspect 9. Aspect 9 is a method comprising administering the composition of Aspect 8to a subject.

Aspect 10. Aspect 10 is the method of Aspect 9, wherein the subject is a human or an animal.

Aspect 11. Aspect 11 is the method of Aspect 9 or 10, wherein the method further comprises treating or preventing a fungal infection, or a biofilm produced from a fungal infection, or a combination thereof in the subject.

Aspect 12. Aspect 12 is the method of any one of Aspects 9 through 11, wherein the fungal infection comprises Candida albicans, Candida albicans, Candida auris, Candida tropicalis, Candida glabrata, Candida krusie, Candida parapsilosis, Rhodotorula mucilaginosa, Rhodotorula minuta, or Rhodotorula glutinis, Cryptococcus neoformans, or Cryptococcus gattii, or any combination thereof.

Aspect 13. Aspect 13 is the method of any one of Aspects 9 through 12, further comprising administering an additional antifungal compound.

Aspect 14. Aspect 14 is the method of any one of Aspects 9 through 13, wherein the administering of the additional antifungal compound occurs at the same time as the administering of the composition.

Aspect 15. Aspect 15 is the method of any one of Aspects 9 through 14, wherein the method further comprises treating or preventing a bacterial infection, or a biofilm produced from a bacterial infection, or a combination thereof, in the subject.

Aspect 16. Aspect 16 is the method of any one of Aspects 9 through 15, further comprising administering an additional antifungal compound.

Aspect 17. Aspect 16 is the method of any one of Aspects 9 through 16, wherein the administering of the additional antifungal compound occurs at the same time as the administering of the composition.

Aspect 18. Aspect 18 is the method of any one of Aspects 9 through 17, wherein the bacterial infection comprises a gram-positive or a gram-negative bacterium.

Aspect 19. Aspect 19 is the method of any one of Aspects 9 through 18, wherein the gram-positive bacterium comprises Enterococcus faecium, Staphylococcus aureus, or Enterococcus faecalis.

Aspect 20. Aspect 20 is the method of any one of Aspects 9 through 19, wherein the gram-negative bacterium comprises Pseudomonas aeruginosa, Enterobacter, Klebsiella pneumoniae, Escherichia coli, or Acinetobacter baumannii.

Aspect 21. Aspect 21 is the method of any one of Aspects 9 through 20, wherein the bacterial infection comprises tuberculosis.

Aspect 22. Aspect 22 is a method comprising: administering the composition of any one of Aspect 8-14 to a plant, a seed of a plant, or a soil the plant grows in.

Aspect 23. Aspect 23 is the method of Aspect 22, wherein the method further comprises treating or preventing a fungal infection, or a biofilm produced from a fungal infection, or a combination thereof, in the plant.

Aspect 24. Aspect 24 is the method of Aspect 22 or 23, wherein the method further comprising administering an additional antifungal compound.

Aspect 25. Aspect 25 is the method of any one of Aspects 22 through 24, wherein the administering of the additional antifungal compound occurs at the same time as the administration of the composition.

Aspect 26. Aspect 26 is the method of any one of Aspects 22 through 25, wherein the fungal infection includes Rhizoctonia solani, Sphaeropsis, Phoma clematidina, Peronosporaceae, Plasmodiophora brassicae, Diplocarpon rosae, Pythium, Phytophthora, Colletotrichum, Gloeosporium, Sclerotinia homoeocarpa, Physoderma, Laetisaria fuciformis, Serpula lacrymans, Synchytrium endobioticum, Ascomycota, Phytophthora infestans, Alternaria solani, Fusarium oxysporum, Verticillium longisporum, Taphrina deformans, Botrytis, Guignardia bidwellii, Venturia inaequalis, Pleurotus ostreatus, Sclerotium rolfsii, Fibroporia vaillantii, Phoma terrestris, Monilinia oxycocci, Ustilago maydis, Phytophthora, Coniophora puteana, Poria vaillantii, Chaetomium, Ceratocystis, or Pyrenophora tritici-repentisa.

Aspect 37. Aspect 27 is the compound or composition of any one of Aspects 1 through 8, wherein a second minimum inhibitory concentration is no greater than a 200% of a first minimum inhibitory concentration, wherein the first minimum inhibitory concentration is evaluated without a serial gain of resistance assay against a microbe of interest, and the second minimum inhibitory concentration is evaluated after the serial gain of resistance assay against the microbe of interest.

Aspect 38. Aspect 38 is the compound or composition of Aspect 37, wherein the microbe of interest is C. albicans.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

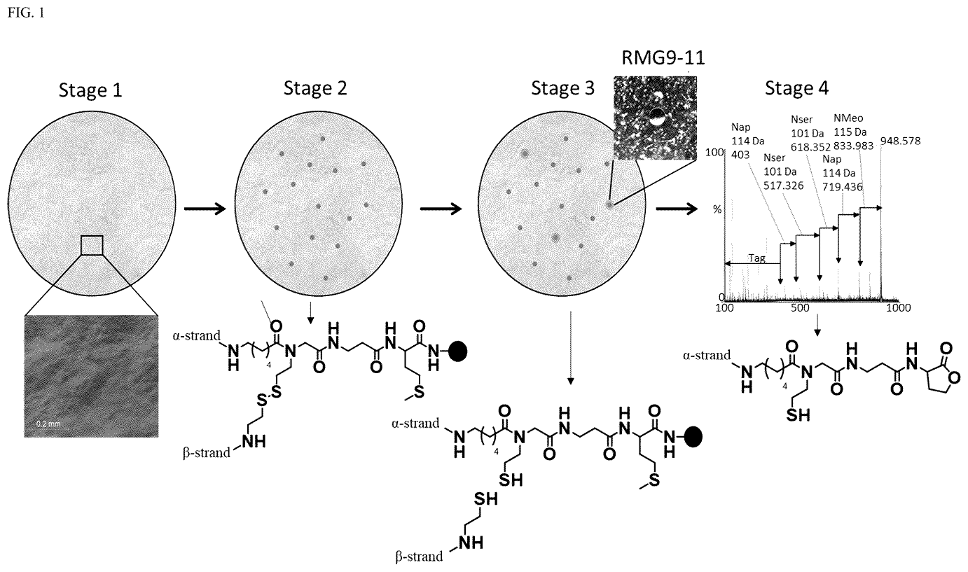

The iPLAD assay was used to discover the exemplar peptoid RMG9-11. The iPLAD assay can be understood in four stages as shown in FIG. 1. In the first stage, an overnight culture of C. albicans in RPMI-MOPS produced a mature biofilm on the surface of a TC treated Petri dish. In the second stage, media was removed, and the biofilm was washed with PBS before a mixture of PLAD linked peptoid library beads and reducing reagent in soft agar liquified at 42° C. was added on top of the biofilm and allowed to solidify. A minimal amount of agar was used to provide good contact between library beads and biofilm. In the third stage, overnight incubation allowed the reducing reagent to release the (β-strand peptoid from library beads which the interacted with the biofilm. Phloxine B was added on top of the soft agar and allowed to diffuse into the biofilm, revealing peptoids that killed fungal cells within the biofilm visualized by a pinkish ring located around “hits”. Finally, in stage four, hits were isolated and the structure of the α-strand was deconvoluted by tandem mass spectrometry.

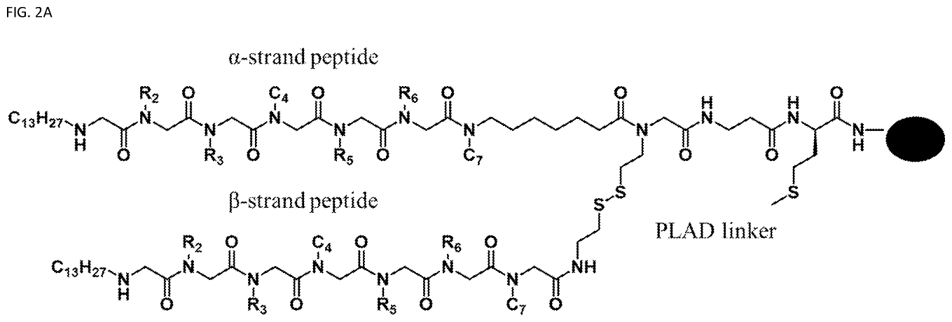

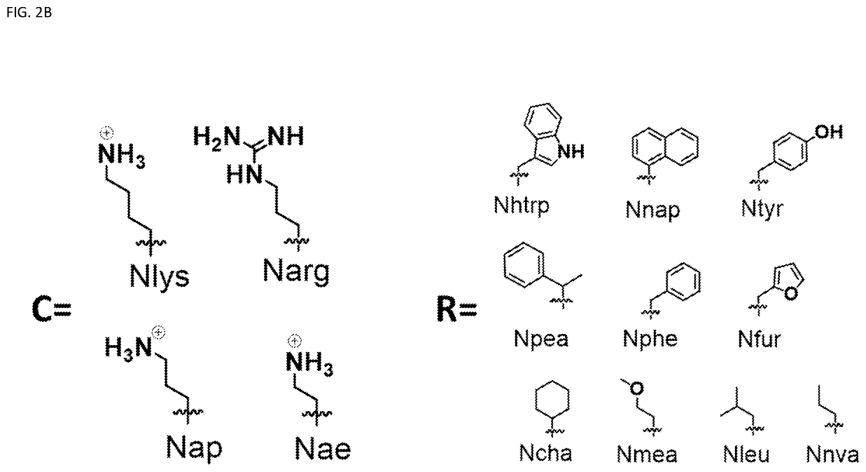

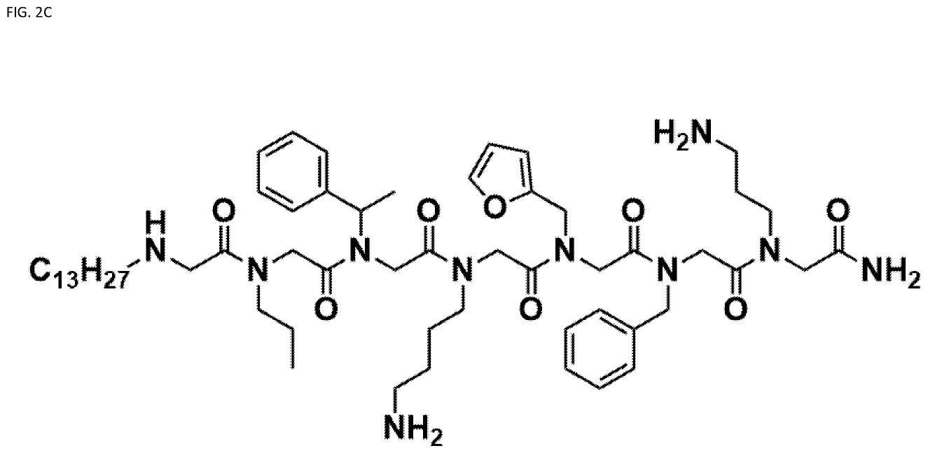

Once optimized, the iPLAD assay was used to screen combinatorial peptoid library RGL9 against C. albicans biofilms. Combinatorial libraries are advantageous as a source of lead molecules since a large collection of unique and diverse compounds can be synthesized and screened rapidly (Lam et al., Chem. Rev. 97, 411-448 (1997)). The design for peptoid library RGL9 has the general sequence of Ntri-N_{R}-N_{R}-N_{C}-N_{R}-N_{R}-N_{C}-linker where Ntri is a tridecylamine lipid tail, N_{R} are randomized aliphatic and aromatic monomers, and N_{C} are randomized cationic monomers (FIG. 2A and FIG. 2B). Addition of a tridecyl amine tail at the N-terminus of combinatorial libraries to yield lipopeptoids is a strategy used to reduce library length and improve hit sequencing by MS/MS (Turkett et al., ACS Comb. Sci. 19, 229-233 (2017); Chongsiriwatana et al., Antimicrob. Agents Chemother. 55, 417-420 (2011)). Through the addition of all these monomers, RGL9 had a theoretical diversity of roughly 1.6×10⁵ unique compounds. This theoretical diversity was limited by the amount of resin used during synthesis, which was 0.5 g, giving an actual diversity for RGL9 of approximately 3.2×10⁴ unique peptoids.

To screen RGL9 using the iPLAD assay, C. albicans mature biofilms were generated by inoculating RPMI-MOPS with a fungal cell solution and incubating overnight. Biofilms were washed gently with PBS to remove any planktonic or dead C. albicans cells. RPMI-MOPS soft agar with 14 mM TCEP and 2-5 mg aliquots of RGL9 resin equilibrated in PBS were added to the washed biofilms and incubated overnight. To determine cell viability and identify hits, phloxine B (5 μM) was added on top of the soft agar and incubated for one hour. Any beads with red halos formed by dead cells unable to efflux phloxine B were considered “hits” and were manually removed, placed into individual tubes, and cleaned by boiling in 1% sodium dodecylsulfate solution to remove media and cellular debris. The α-strand peptoid was then cleaved from the bead using cyanogen bromide and analyzed by tandem mass spectrometry to determine the unknown peptoid sequence.

This screening led to the discovery of RMG9-11 (FIG. 2C). The minimum inhibitory concentration (MIC) of resynthesized RMG9-11 was initially determined against C. albicans, C. neoformans, and the ESKAPE bacteria via CSLI guidelines (CLSI. Methods for Dilution Antimicrobial Susceptibility Tests for Bacteria That Grow Aerobically, 11th Edition. Clinical and Laboratory Standards Institute (2018); CLSI. Reference method for broth dilution antifungal susceptibility testing of yeasts; approved standard-third edition; CLSI document M27-A3. Clinical and Laboratory Standards Institute (2008)). Table 1 shows the minimum inhibitor concentration (MIC) values of RMG9-11 against fungal and bacterial pathogens. C. albicans clinical isolates include a reference strain (M1), fluconazole susceptible strains (M4 and M6), and fluconazole resistant strains (M2, M3, M5, and M7). ATCC 64124 is multidrug resistant C. albicans strain that is resistant to amphotericin B, fluconazole, caspofungin, and flucytosine. All MICs were performed under CSLI guidelines in triplicate on at least three different days.

The MIC for RMG9-11 against C. albicans was 6.25 μg/mL, which is comparable to other antifungal peptoids (Chongsiriwatana et al., Antimicrob. Agents Chemother. 55, 417-420 (2011)) and several clinical treatments for this fungal pathogen, including fluconazole and flucytosine (Pinto et al., Microbiol. Res. 163, 579-585 (2008)). As observed with previously discovered antifungal peptoids, RMG9-11 was more effective at killing C. neoformans than C. albicans, (Spicer et al., Biopolymers 110, e23276 (2019); Ryge et al., Chemotherapy 54, 152-156 (2008); Green et al., Int. J. Antimicrob. Agents 56, 106048 (2020)) although only at a 2-fold improvement from 6.25 to 3.13 μl g/mL, respectively. The biofilm MIC (BMIC) of RMG9-11 increased 16-fold compared to the MIC (BMIC=100 μg/mL). BMIC is often elevated compared to MIC for a number of reasons, including a 100-to 1000-fold increase in cell number for the established biofilm. Other theories suggest that the negatively charged polysaccharide matrix of the biofilm can bind up cationic antimicrobial peptides and peptoids, preventing their engagement of the pathogen target Yasir et al., Materials 11, (2018)).

TABLE 1
MIC of RMG9-11 against a variety of fungi and bacterial strains.

| Pathogen/Strain | MIC (μg/mL) |
|---|---|
| C. albicans | 6.25 |
| C. albicans biofilm | 100 |
| C. albicans M1 | 6.25 |
| C. albicans M2 | 12.5 |
| C. albicans M3 | 12.5 |
| C. albicans M4 | 12.5 |
| C. albicans M5 | 12.5 |
| C. albicans M6 | 6.25 |
| C. albicans M7 | 6.25 |
| C. albicans ATCC 64124 | 6.25 |
| C. auris | 12.5 |
| C. tropicalis | 3.13 |
| C. glabrata | 6.25 |
| C. krusie | 3.13 |
| C. parapsilosis | 6.25 |
| C. neoformans | 3.13 |
| E. faecalis | 6.25 |
| E. faecium | 3.13 |
| S. aureus | 6.25 |
| K. pneumonia | 50 |
| A. baumannii | 12.5 |
| P. aeruginosa | 12.5 |
| E. coli | 6.25 |
| M. smegmatis | 6.25 |

RMG9-11 was evaluated against fluconazole resistant and susceptible strains of C. albicans ith MICs between 6.25-12.5 μg/mL regardless of fluconazole susceptibility. Encouragingly, RMG9-11 maintained potency against ATCC 64124 (6.25 μg/mL), a C. albicans strain with amphotericin B, caspofungin, fluconazole, and flucytosine drug resistance. The potency of RMG9-11 against C. auris was investigated. C. auris was identified in 2007 as a multidrug resistant strain of Candida and was observed first in Japan followed by cases in Africa and Europe (Kathuria et al., J. Clin. Microbiol. 53, 1823-1830 (2015); Borman et al., mSphere 1, 4-6 (2016); Borman et al., mSphere 1, 4-6 (2009); Magobo et al., Emerg. Infect. Dis. 20, 1250-1251 (2014)). Only a modest decrease in efficacy was seen for RMG9-11 with C. auris (12.5 μg/mL), which was encouraging given that C. auris is resistant to most clinical antifungal agents (Du et al., PLOS Pathog. 16, e1008921 (2020)). Other common Candida species (tropicalis, krusei, glabrata, and parapsilosis) were evaluated to determine if RMG9-11 could be used as a pan-Candida treatment. RMG9-11 appears promising with MIC values ranging from 3.13 to 6.25 μg/mL against all Candida species tested, maintaining or even improving efficacy compared to C. albicans. The efficacy of RMG9-11 was evaluated against bacterial pathogens, specifically the ESKAPE bacteria, to determine if RMG9-11 could be used on a wider scale.

The ESKAPE bacteria consist of the most common multidrug resistant and nosocomial bacterial pathogens including both Gram-positive and Gram-negative species (Pendleton et al., Expert Rev. Anti-infect. Ther. 11, 297-308 (2013)). Encouragingly, RMG9-11 had moderate activity against both Gram-negative and Gram-positive bacteria (3.13-12.5 μg/mL; FIG. 3), with the exception of Gram-negative K. pnuemoniae (50 μg/mL). The broad-spectrum antimicrobial activity of RMG9-11 naturally lead to concerns about mammalian cytotoxicity, especially since the membranes of fungi and mammals have similar phospholipid compositions.

The mammalian cytotoxicity of RMG9-11 was evaluated (Table 2). Table 2 shows the cytotoxicity analysis of RMG9-11 against several different mammalian cell lines. The peptoid concentrations that resulted in a 50% reduction in viable cells (TD₅₀; toxicity dose 50%) for liver (HepG2), fibroblast (3T3), and keratinocytes (HaCat) are shown. Also shown is the peptoid concentration that resulted in 10% hemolysis (HC₁₀) of single donor human red blood cells (hRBC). Selectivity ratios (SR) were calculated as cytotoxicity divided by MIC against planktonic C. albicans.

The mammalian cell lines that were tested (HepG2, 3T3, and HaCat) were incubated with RMG9-11 for 72 hours and cell viability was determined via mitochondrial activity using an MTT reduction assay where viable cells reduce MTT to formazan. Toxicity is reported as the concentration of the peptoid that results in a 50% reduction in viable cells, termed toxicity dose 50% or TD₅₀. The cytotoxicity of RMG9-11 was first determined against HepG2 hepatocellular carcinoma cells with a TD₅₀ of 114 μg/mL and no toxicity at the MIC against C. albicans, giving a modest selectivity ratio (SR) of 18 (Table 2). SR is defined as the TD₅₀ divided by MIC and provides a measure of the therapeutic window for a compound. Typically for most lead compounds that would continue through development and optimization, a SR of 10 is preferred while an SR of 100 is more desirable for a compound moving into preclinical animal model evaluation. RMG9-11 displayed increased toxicity against mouse fibroblasts (3T3; TD₅₀=39 μg/mL) and keratinocytes (HaCat; TD₅₀=53 μg/mL), though toxicity was still minimal at the MIC. Hemolytic analysis showed RMG9-11 to be moderately toxic to hRBCs with a HC₁₀ of 29 μg/mL and a SR of 5.

TABLE 2
Mammalian cytotoxicity of RMG9-11.

| Cell | TD₅₀ | HC₁₀ | |
|---|---|---|---|
| Line | (μg/mL) | (μg/mL) | SR |
| HepG2 | 114 ± 5 | — | 18 |
| 3T3 | 39 ± 2 | — | 6 |
| HaCat | 53 ± 5 | — | 8 |
| hRBC | — | 29 ± 2 | 5 |

One of the most serious crises facing both new and existing antimicrobial compounds is the rapid development of resistance by pathogens to that compound, rendering it useless. This has been an issue observed with all major commercially available antimicrobial drugs and is an increasingly prevalent issue with antifungal drugs (Clatworthy et al., Nat. Chem. Biol. 3, 541-548 (2007); Wiederhold et al., Infect. Drug Resist. 10, 249-259 (2017)). The ability of C. albicans to gain resistance against RMG9-11 was evaluated using two techniques; serial and single passage. Ching et al. described a method for single passage assessment of gain of resistance, which was used here with RMG9-11 (Ching et al., Sci. Rep. 10, 8754 (2020)). Briefly, a single colony of C. albicans was incubated overnight in RPMI-MOPS. Fungal cultures were then incubated for either 24 or 48 hours with concentrations of RMG9-11 ranging from 0 to 80% MIC. Fungi exposed to all concentration of RMG9-11 for either 24 and 48 hours were still equally susceptible to peptoid in a standard MIC assay (MIC=6.25 μg/mL; Table 3). These findings suggest that C. albicans is unable to develop resistance to RMG9-11. To further solidify this finding, C. albicans were introduced to increasing concentrations of RMG9-11 through a serial passage gain of resistance assay, as described by Samuelsen et al. (Samuelsen et al., FEBS Lett. 579, 3421-3426 (2005)). Initially, C. albicans were grown in peptoid free media and in successive days introduced to increasing concentrations of RMG9-11 ranging from 1 to 25% MIC. One main difference between serial and single passage assays is in the inoculation method from one passage to the next. The single passage relies on one individual colony while the serial passage focuses on a set concentration of fungi (cells/mL). At the end of serial passage, C. albicans that had been exposed to gradually increasing concentrations of RMG9-11 were used to prepare a traditional MIC assay. In theory, if fungi had evolved resistance during serial passage with sub-MIC antifungal agent, the MIC of RMG9-11 against these fungi should increase. This alternative approach to microbial gain of resistance had the same result as the single passage method with RMG9-11 maintaining a MIC of 6.25 μg/mL (Table 3). Overall, these data indicate that C. albicans is unable to rapidly develop resistance to RMG9-11, validating the promise of this peptoid as a potential lead antifungal agent. To our knowledge, this is the first evaluation of the ability of a microbe to gain resistance to a peptoid, though it has long been hypothesized that gaining resistance to peptoids is unlikely.

TABLE 3
MIC values for C. albicans gain of resistance against RMG9-11.

| | |
|---|---|
| Single Passage | Serial Passage |

| | 24 h MIC | 48 h MIC | | MIC |
|---|---|---|---|---|
| % MIC | (μg/mL) | (μg/mL) | | (μg/mL) |
| 0 | 6.25 | 6.25 | Untreated | 6.25 |
| 10 | 6.25 | 6.25 | Treated | 6.25 |
| 20 | 6.25 | 6.25 | | |
| 40 | 6.25 | 6.25 | | |
| 60 | 6.25 | 6.25 | | |
| 80 | 6.25 | 6.25 | | |

The antifungal efficacy, mammalian cytotoxicity, and selectivity ratio of RMG9-11 can be improved through an iterative structure activity relationship (SAR) study (Middelton et al., Bioorg. Med. Chem. Lett. 28, 3514-3519 (2018)). A series of proposed RMG9-11 derivatives are shown in FIG. 3. This list of derivatives is not exhaustive. A broad range of derivatives may be explored, guided by an initial sarcosine scan to determine the pharmacophoric importance of each peptoid monomer.

### Abbreviations

iPLAD, Inverted Peptoid Library Agar Diffusion; AMP, antimicrobial peptide; YPD, yeast extract peptone dextrose; MIC, minimum inhibitory concentration; TD₅₀, toxicity dose 50%; HC₁₀, hemolytic concentration 10%; SR, selectivity ratio.

### Experimental Methods

### Materials

Reagents were purchased from Fisher Scientific (Waltham, MA), Alfa Aesar (Haverhill, MA), Amresco (Solon, OH), TCI America (Portland, OR), Anaspec (Fremont, CA), EMD Millipore (Billerica, MA), Peptides International (Louisville, KY), and Chem-Implex (Wood Dale, IL). All reagents used were greater than 95% purity. Human red blood cells (hRBCs) were acquired from Innovative Research (Novi, MI). Boc and Mmt protected diamines were purchased from Chem-Impex (Wood Dale, IL) or synthesized as previously described .44 Microscope images were captured using a Leica M165FC microscope. All mass spectra were acquired on a Waters Synapt HDMS QToF with Ion Mobility. All fluorescence and absorbance readings were acquired on a Spectramax M5 plate reader. Purification of compound was achieved by Varian Prepstar SD-1 with Supelco Ascentis C18 column (511.M; 25 cm×21.2 mm; Sigma-Aldrich 581347-U) and a 0-100% gradient of water to acetonitrile containing 0.05% trifluoroacetic acid.

### RGL9 Library Synthesis

The PLAD linker was synthesized as previously described (Fischer et al., ACS Comb. Sci. 18, 287-291 (2016)). RGL9 was synthesized using a split-and-pool method onto TentaGel containing the PLAD linker. Briefly, resin (500 mg) was bromoacylated using bromoacetic acid (BrAcOH; 2 M) with diisopropylcarbiimidine (DIC; 3.2 M) in dimethylformamide (DMF) and agitated for 10 minutes after microwave assistance (10% power; 15 seconds; 2×). Resin was then evenly divided into either four cationic or ten noncationic reaction vials and treated with amine solutions (2 M) in DMF and agitated for 45 minutes after microwave assistance. Resin was then pooled together and the entire bromoacylation, split, amination, pool process repeated six times. Positions four and seven were reserved for cationic submonomers and positions two, three, five, and six were reserved for noncationic submonomers/The amines used during split-and-pool synthesis were Boc-ethylenediamine, Boc-diaminopropane, Boc-diaminobutane, Mmt-diaminopropane, homotryptamine, furfurylamine, 4-(aminomethyl) phenol, benzylamine, cyclohexylamine, methoxyethylamine, naphthylamine, isopropylamine, (+/−) phenylethylamine, and propylamine. This gave RGL9 a theoretical diversity of 1.6×107. After split-and-pool synthesis was complete, a final addition of tridecylamine was completed using peptoid chemistry. The terminal amine was then Boc-protected using Di-tert-butyl dicarbonate (Boc₂O) in 5% N-methylmorpholine (NMM) in DMF at 10 molar equivalents compared to resin loading capacity. Amines intended for guanidinylation to form arginine mimics were Mmt-deprotected using trifluoracetic acid (TFA; 1%) in dichloromethane and agitated with resin (5×, 10 minutes). Guanidylation was achieved by agitating library overnight with pyrazole carboxamidine (10 molar equivalents) with 4-dimethylaminopyridine (DMAP; 1 molar equivalent) in 5% NMM-DMF. Global Boc deprotection was achieved by treating with TFA, water, and triisopropylsilane (TIS) at a 95:2.5:2.5 ratio, respectively for 1 h followed by washing with dichloromethane to yield the final RGL9 peptoid library.

### iPLAD Assay

Yeast Peptone Dextrose (YPD) agar plate was streaked with frozen stocks of C. albicans and incubated overnight at 37° C. Single colonies were added to saline solution (0.85%) until a turbidity between OD₅₃₀=0.15-0.25 was achieved. Cell solution (100 μL) was added to RPMI-MOPS (pH 7; 6 mL) and plated onto a cell-treated Petri plate (60 mm) and incubated overnight at 37° C. Media was removed and biofilms were washed gently with PBS (3×). RPMI-MOPS soft agar (0.75% w/v) was liquefied and maintained at 42° C. until use. RGL9 (2-5 mg in 0.5 mL PBS) and TCEP (100 mM; 580 μL) was added to soft agar (4 mL total volume) and plated on top of biofilm. Plates were incubated at 37° C. overnight. Phloxine B (10 μg/mL; 1 mL) was added on top of the soft agar and incubated for an additional 1 hour. Beads with red halos were extracted with surgical tweezers, placed in individual microcentrifuge tubes, and boiled in sodium dodecyl sulfate (SDS; 1%) for 10 minutes followed by washing with PBS (3×). The remaining α-strand peptoid on these beads was cleaved from resin by incubating with cyanogen bromide (40 mg/mL) in acetonitrile: water (1:1) with hydrochloric acid (0.1 M) overnight at room temperature in the dark. The cyanogen bromide solution was removed in vacuo and resuspend in acetonitrile: water (1:1) with TFA (0.05%) and analyzed via MS and sequenced using MS/MS to obtain structures of the unknown peptoids.

### Peptoid Synthesis

Peptoid sequences determined by MS/MS were re-synthesized for antimicrobial characterization using the standard submonomer approach (Zuckermann et al., J. Am. Chem. Soc. 114, 10646-10647 (1992)). Peptoids were synthesized on polystyrene (PS) rink amide resin. Resin was swelled in DMF for 20 minutes. Resin was Fmoc-deprotected using 20% piperidine in DMF and agitated for 10 minutes twice. Removal was confirmed by colorimetric Kaiser test. Resin was bromoacylated using BrAcOH (2 M) and DIC (3.2M) in anhydrous DMF and agitated for 10 minutes following microwave assistance. After washing with DMF, amines were coupled by agitating resin with amine of choice (2 M) in anhydrous DMF for 25 minutes after microwave assistance followed by washing with DMF. This process was repeated to form the desired peptoid. Peptoids were cleaved from resin and Boc groups simultaneously deprotected by agitating with TFA, water, TIS (95:2.5:2.5) for one hour. TFA solution was removed by bubbling the solution with air followed by resuspension in acetonitrile: water (1:1) with TFA (0.05%) and verification by MS. Peptoids were then purified using RP-HPLC with a SUPLRCO C-18 column and a water to acetonitrile (0 to 100%) gradient containing 0.05% TFA. Solvent was removed in vacuo to yield peptoids as white powders.

### Fungal Minimum Inhibitory Concentration Assay

The minimum inhibitory concentration (MIC) of peptoids against fungal pathogens C. albicans and C. neoformans was determined following CLSI guidelines (Reference method for broth dilution antifungal susceptibility testing of yeasts; approved standard-third edition; CLSI document M27-A3.” CLSI. 2008.). Colonies were transferred from a streaked YPD plate to 0.85% saline to reach an OD₅₃₀ between 0.18 and 0.25. This inoculant was diluted 1:100 into RPMI-MOPS and then further diluted 1:20 into RPMI-MOPS. 198 μL of inoculant was seeded into each well of a 96 well black-walled plate. 2-fold serial dilutions of 100× peptoid solutions were prepared in water and 2 μL of peptoid were added to each well in triplicate. Plate was incubated at 37° C. for 72 hours for C. neoformans and 24 hours for C. albicans before evaluating the MIC by manual observation. MIC was defined as the lowest concentration of compound preventing fungal growth. This assay was repeated 3 times on separate days with each compound.

### Fungal Biofilm Minimum Inhibitory Concentration Assay

The biofilm minimum inhibitory concentration (MIC) of peptoids against fungal pathogens C. albicans was determined following CLSI guidelines (CLSI. Reference method for broth dilution antifungal susceptibility testing of yeasts; approved standard-third edition; CLSI document M27 A3. Clinical and Laboratory Standards Institute, 2008). Colonies were transferred from a streaked YPD plate to 0.85% saline to reach an OD₅₃₀ between 0.15 and 0.25. This inoculant was diluted 1:100 into RPMI-MOPS and then further diluted 1:20 into RPMI-MOPS. 200 μL of inoculant was seeded into each well of a 96 well black-walled plate. Plates were incubated overnight at 37° C. Media was gently removed and gently washed three times with PBS. RPMI-MOPS (198 μL) were added to each well. 2-fold serial dilutions of 100× peptoid solutions were prepared in water and 2 μL of peptoid were added to each well in triplicate then incubated at 37° C. for 24 hours. PRESTOBLUE (20 μL) was added to each well and incubated at 37° C. for one hour before measuring fluorescence on a SPECTRAMAX M5 plate reader (Ex. 555 nm; Em. 585 nm).

### Bacterial Minimum Inhibitory Concentration Assay

The minimum inhibitory concentration (MIC) of peptoids against the ESKAPE bacteria (Enterococcus faecium ATCC 6569; Staphylococcus aureus ATCC 29213; Klebsiella pneumoniae ATCC 13883; Acinetobacter baumanii ATCC 19606; Pseudomonas aeruginosa ATCC 25619; Enterococcus faecalis ATCC 29212; and Escherichia coli ATCC 25922) was determined following CLSI guidelines (CLSI. Methods for Dilution Antimicrobial Susceptibility Tests for Bacteria That Grow Aerobically, 11th Edition. Clinical and Laboratory Standards Institute, 2018). The MIC against Mycobacterium smegmatis was also determined following CLSI guidelines (Woods, G. L. et al. Susceptibility Testing of Mycobacteria, Nocardiae, and Other Aerobic Actinomycetes, 2nd edition. (2011)). Colonies picked from streaked tryptic soy agar plates were added to tryptic soy broth (TSB) to achieve a turbidity of OD₆₀₀=0.08-0.15. Inoculant was diluted 1:200 into cation adjust Mueller Hinton Broth (CAMHB) and 90 μL was plated into each well of a 96 well black-wall plate. 2-fold serial dilutions of 10× peptoid (10 μL) were added to each well in triplicate and incubated at 37° C. for 24 hours for the ESKAPE bacteria and 72 hours for M. smegmatis. Tetracycline (20 μg/mL) was used as a positive control with DI water was used as a vehicle control. Following incubation for the ESKAPE bacteria, PRESTO BLUE (10 μL) was added to each well and incubated at 37° C. for one hour before measuring fluorescence on a SPECTRAMAX M5 plate reader (Ex. 555 nm; Em. 585 nm). Following incubation for M. smegmatis, wells were scored following CLSI guidelines to determine MIC. This assay was repeated three times on separate days.

### Mammalian Cytotoxicity

Hepatocellular carcinoma (HepG2), mouse fibroblast (3T3), and human keratinocyte (HaCat) cells were cultured in Dulbeco modified eagle media (DMEM) containing 10% fetal bovine serum (FBS) and 1% penicillin, streptomycin, and glutamine (PSG) at 37° C. and 5% CO₂. Cells were collected and adjusted to 1×105 to 4×105 cells/mL in phenol-red free DMEM and plated (100 μL) 96 well plates. 2-fold serial dilutions of 10× peptoid (11.1 μL) were added to each well in triplicate. Water (vehicle) was used as a negative control. Plates were incubated at 37° C. in 5% CO₂ for 72 hours. Thiazolyl blue tetrazolium bromide (MTT) was added to each well (5 mg/mL; 20 μL) and incubated for 3 hours. Media was removed, DMSO (100 μL) was added, and plates were incubated at 37° C. for 10 minutes. Plates were read on a SPECTRAMAX M5 plate reader (Abs. 570 nm). The concentration of compound resulting in a 50% reduction in growth compared to control (toxicity dose 50%; TD₅₀) was determined using GRAFIT. This procedure was repeated three times on separate days.

### Hemolytic Assay

Hemolytic activity was determined using single donor human red blood cells (hRBC). hRBCs were washed with PBS and centrifuged (1000 RPM; 10 minutes) three times, resuspended in PBS and aliquoted (100 μL) in 96 well plates. 2-fold serial dilutions of 10× peptoid final concentrations in PBS were prepared and added to wells in triplicate. PBS was used as a vehicle control and 1% Triton X-100 as a positive control. Plates were incubated for 1 hour (37° C.; 5% CO2) then centrifuged (1000 RPM; 10 minutes) and supernatant was diluted 1:20 into PBS in a new 96-well plate. Plates were read on a SPECTRANAX M5 plate reader (Abs. 405 nm). Percent hemolysis was determined by the following equation:

$$\\ \% hemolysis = \frac{{OD}_{405 nm} sample - {OD}_{405 nm} 405 negative control}{{OD}_{405 nm} positive control - {OD}_{405 nm} negative control} \times 100$$

GRAFIT was used to determine concentrations at 50% hemolytic activity (HC50) and the Hill coefficient (H). Hemolytic activity at 10% (HC₁₀) was then determined by the following equation:

$$\\ {HC}_{10} = {{HC}_{50} [ 10 \% / ( 100 \% - 10 \% ) ]}^{1 / H}$$

### Serial Passage Gain of Resistance Assay

Gain of resistance to RMG9-11 by C. albicans was determined using a serial passage assay following previously published methods.43 Single colonies of C. albicans from a frozen stock streak plate were added to a saline solution (0.85%) to achieve an OD₅₃₀=0.15-0.25. RPMI-MOPS (pH 7; 5 mL) was inoculated with cell solution to reach a concentration of 105 cell/mL (OD₅₃₀ of 1.000=3×107 cells/mL). RMG9-11 was added at 1% MIC and incubated overnight (200 RPM; 37° C.). Fresh RMPI-MOPS was inoculated with overnight cell solution containing a 5% MIC concentration of peptoid. This process was repeated, increasing from 5 to 10 and finally to 25% MIC sequentially. A MIC was performed after 25% MIC incubation following CLSI guidelines (CLSI. Reference method for broth dilution antifungal susceptibility testing of yeasts; approved standard-third edition; CLSI document M27-A3. Clinical and Laboratory Standards Institute, 2008) using cells from the serial passage culture. This was performed with four separate streaked plates from frozen stocks for biological replicate.

### Single Passage Gain of Resistance Assay

A second gain of resistance through single passage was done for RMG9-11 against C. albicans following previously published methods as a confirmation of serial passage results (Ching et al., Sci. Rep. 10, 8754 (2020)). A single C. albicans colony was inoculated into RPMI-MOPS (pH 7; 5 mL) and incubated overnight (200 RPM; 37° C.). Overnight solution was diluted 1:1000 into RPMI-MOPS (pH 7; 5 mL) containing 0, 10, 20, 40, 60, and 80% MIC concentrations of RMG9-11 and incubated for 24 hours (200 RPM; 37° C.). Overnight solution was either diluted 1:1000 into YPD or fresh RPMI-MOPS containing 0, 10, 20, 40, 60, or 80% MIC RMG9-11 and incubated for an additional 24 hours. YPD solutions were used to setup a traditional MIC assay to evaluate gained resistance after 24 hours while 48-hour RPMI-MOPS solutions were diluted 1:1000 into YPD without compound and incubated overnight. The following day, an MIC was performed with these cell solutions to evaluate gain of resistance after 48 hours. This assay was performed with four separate streaked plates from frozen stocks for biological replicate.

### Example 2

### RMG9-11 Sarcosine Scan

An iterative structure-activity relationship (SAR) of RMG9-11 was done to explore derivatives of this compound with the goal of developing a derivative with improved biological activity and selectivity. Initially, a sarcosine scan was completed to determine the pharmacological importance of each monomer. In this study, derivatives were synthesized where each monomer of RMG9-11 was substituted with sarcosine, or N-methylglycine, which is the peptoid equivalent of alanine (FIG. 5). The antifungal efficacy (minimum inhibitory concentration or MIC) against Candida albicans and the mammalian cytotoxicity (toxicity does 50% or TD₅₀) against HepG2 liver cells was determine for each compound (Table 4).

TABLE 4
MIC and TD₅0 of RMG9-11 sarcosine scan compounds.

| Compound - group | MIC | TD₅₀ |
|---|---|---|
| replaced with sarcosin | (μg/mL) | (μg/mL) |
| RMG9-11 | 6.25 | 114 ± 5 |
| SAR 1 - tridecylamine | >100 | >200 |
| SAR 2 - propylamine | 12.5 | 83.9 ± 5.7 |
| SAR 3 - phenylethylamine | 50 | 194.5 ± 7.5 |
| SAR 4 - diaminobutane | 6.25 | 61.8 ± 16.1 |
| SAR 5 - furfurlyamine | 12.5-25 | 156.6 ± 10.1 |
| SAR 6 - benzylamine | 25 | 155.5 ± 4.7 |
| SAR 7 - diaminopropane | 6.25 | 57.7 ± 11 |

These data indicated that the lipophilic tridecyl tail in position 1 (position T of the general Formula I) was pharmacologically important, contributing the most to both antifungal efficacy and cytotoxicity. Second was the phenylethyl group (Npea) in position 3 (R²), followed by the benzyl group (Nphe) in position 6 (R⁵), the furfuryl group (Nfur) in position 5 (R⁶), the propyl group (Nnva) in position 2 (R¹), and the cationic groups (Nlys and Nap) in positions 4 (R³) and 7 (R⁶). The cationic groups primarily mitigate cytotoxicity and contribute very little if at all to antifungal efficacy. Additionally, the propyl group in position 2 (R¹) contributed very little to either antifungal efficacy or cytotoxicity and it was hypothesized that this site could be modified without much biological penalty to development.

### Iterative SAR Round 1

Round 1 of the iterative SAR explored two miscellaneous derivatives along with derivatives that explored substitutions in position 3 (R²; FIG. 6). Structures were confirmed by ESI-MS and hydrophobicity was determined by the percent acetonitrile at time of elution during RP-HPLC purification and using MarvinSketch to calculate the distribution coefficient at pH 7.4 (Table 5). Only one derivative of position 1 (group T of Formula 1) was explored, substituting a 14-carbon fatty acid (myristic acid) tail in place of the tridecyl tail (911-M1). This was tested because this fatty acid was previously shown to improve activity with C. albicans while reducing toxicity (Green R M, Bicker R. L. Evaluation of peptoid mimics of short, lipophilic peptide antimicrobials. Int J Antimicrob Agents. 2020; 56(2):106048. doi:10.1016/j.ijantimicag.2020.106048). The second miscellaneous derivative tested substituting the propyl group (Nnva) in position 2 (R¹) with a cationic group in an effort to reduce toxicity without effecting antifungal activity (911-M2). Substitutions in position 3 (R²) included a benzyl group (Nphe; 911-M3), a para-fluorobenzyl group (Npfb; 911-M4), a para-chlorobenzyl group (Npcb; 911-M5), a para-bromobenzyl group (Npbb; 911-M6), a furfuryl group (Nfur; 911-M7), a thiophene group (Ntma; 911-M8), a cyclohexyl group (Ncha; 911-M9), a cyclohexylmethyl group (Nchexm; 911-M10), and a 5-cyclohexylethyl group (NchexmS; 911-M11).

TABLE 5
Physicochemical characterization of Round 1 derivatives.
Observed molecular weights determined by ESI-MS. % ACN and
cLogD₇₄ provided as measures of hydrophobicity. % ACN = percent
acetonitrile at time of elution from RP-HPLC. cLogD_{7.4} =
calculated distribution coefficient at pH 7.4 (Chemaxon.
MarvinSketch 19.17.0 was used for physicochemical calculations.
(https://www.chemaxon.com) (2019)).

| | Expected | Observed | % | % | |
|---|---|---|---|---|---|
| Compound | m/z | m/z | yield | ACN | cLogD_{7.4} |
| 911-M1 | 1072.43 | 1072.46 | 39 | 74 | −1.94 |
| 911-M2 | 1045.40 | 1045.12 | 39.5 | 68.3 | −6.11 |
| 911-M3 | 1030.39 | 1030.10 | 12.4 | 71.1 | −3.4 |
| 911-M4 | 1048.38 | 1048.10 | 24.5 | 71.3 | −3.25 |
| 911-M5 | 1064.83 | 1064.08 | 15.7 | 71.5 | −2.79 |
| 911-M6 | 1109.29 | 1110.07 | 9.1 | 71.7 | −2.63 |
| 911-M7 | 1020.35 | 1020.07 | 23.4 | 70.3 | −4.33 |
| 911-M8 | 1036.41 | 1036.06 | 22.4 | 70.8 | −3.48 |
| 911-M9 | 1022.41 | 1022.13 | 17.6 | 71.2 | −3.32 |
| 911-M10 | 1036.44 | 1036.14 | 18.8 | 72.3 | −3.01 |
| 911-M11 | 1050.46 | 1050.17 | 16.2 | 73 | −2.59 |

Antifungal activity was determined for all derivatives against C. albicans and Cryptococcus neoformans and is reported as MIC (Table 6). Mammalian cytotoxicity was determined against HepG2 liver cells and is reported as TD50 (Table 6). These data indicate that compound 911-M1 with the fatty acid tail and compound 911-M11 with the S-cyclohexylethyl group (NchexmS) had modestly improved antifungal activity against C. albicans. Interestingly, compounds 911-M8 and 911-10 displayed sub-one μg/mL activity against C. neoformans. Most of the derivatives in Round 1 had similar or increased cytotoxicity compared to RMG9-11, and thus diminished selectivity ratios (SRs), calculated by dividing the TD₅₀ by the MIC. Compound 911-M2, with the additional cationic group displayed a dramatic decrease in cytotoxicity, but also displayed reduced antifungal activity. The most promising compound from Round 1 was 911-M1, which had both improved antifungal activity and decreased cytotoxicity, resulting in an improved SR. Several promising elements of Round 1 were combined to generate Round 1 hybrids that were hypothesized to have promising biological properties. Specifically, these elements were the myristic fatty acid ((CO)—R²⁰, where R²⁰ is linear (C₁₃)alkyl) in position 1 (group T), the extra cationic group in position 2 (R¹), and the S-cyclohexylethyl group (NchexmS) in position 3 (R²). Hybrids containing all three elements (911-M12) or only the last two elements (911-M13) were synthesized and characterized (FIG. 7 and Table 7). There hybrids did not display increased selectivity compared to 911-M1, therefore, the myristic acid in position 1 (group T) was carried forward into Round 2 of SAR development.

TABLE 6
Antifungal and cytotoxicity data for Round 1 derivatives.
MIC = minimum inhibitory concentration. TD₅₀ =
toxicity dose 50%. SR = selectivity ratio (SR = TD₅₀/MIC).

| | C. | C. | HepG2 | C. | C. |
|---|---|---|---|---|---|
| | albicans | neoformans | TD₅₀ | albicans | neoformans |
| Compound | (μg/mL) | (μg/mL) | (μg/mL) | SR | SR |
| RMG9-11 | 6.25 | 3.13 | 114 ± 5 | 18 | 36 |
| 911-M1 | 3.13-6.25 | 3.13 | 144.5 ± 22.6 | 23-46 | 46 |
| 911-M2 | 6.25-12.5 | 3.13 | 177.3 ± 20.5 | 14-28 | 57 |
| 911-M3 | 6.25 | 1.56 | 87.5 ± 10.8 | 14 | 56 |
| 911-M4 | 6.25 | 1.56 | 119.9 ± 36.9 | 19 | 77 |
| 911-M5 | 6.25-12.5 | 1.56 | 114.4 ± 32.5 | 9-18 | 73 |
| 911-M6 | 6.25 | 1.56-3.13 | 107.5 ± 13.6 | 17 | 34-69 |
| 911-M7 | 12.5 | 3.13 | 187 ± 21.5 | 15 | 60 |
| 911-M8 | 6.25 | 0.78-1.56 | 107.6 ± 34.6 | 17 | 69-138 |
| 911-M9 | 12.5 | 1.56 | 99.5 ± 22 | 8 | 64 |
| 911-M10 | 6.25 | 0.78-1.56 | 97.8 ± 18.3 | 16 | 63-125 |
| 911-M11 | 3.13-6.25 | 1.56 | 82.5 ± 11.5 | 13-26 | 53 |
| RMG 9-11 | 6.25 | 3.13 | 114 ± 5 | 18 | 36 |
| RMG 8-8 | 25 | 1.56 | 189 ± 43 | 8 | 121 |

TABLE 7
Antifungal and cytotoxicity data for Round 1 hybrids. MIC = minimum inhibitory
concentration. TD₅₀ = toxicity dose 50%. SR = selectivity ratio (SR = TD₅₀/MIC).

| | C. | C. | HepG2 | C. | C. |
|---|---|---|---|---|---|
| | albicans | neoformans | TD₅₀ | albicans | neoformans |
| Compound | (μg/mL) | (μg/mL) | (μg/mL) | SR | SR |
| 911-M12 | 6.25 | 3.13 | 145 ± 35 | 23 | 46 |
| 911-M13 | 6.25 | 3.13 | 140 ± 33 | 22 | 45 |
| RMG 9-11 | 6.25 | 3.13 | 114 ± 5 | 18 | 36 |
| 911-M1 | 3.13-6.25 | 3.13 | 144.5 ± 22.6 | 23-46 | 46 |

### Iterative SAR Round 2

Round 2 of the iterative SAR explored modifications of the second most pharmacologically important monomer as determined by the sarcosine scan, the benzyl group (Nphe) in position 6 (R⁵; FIGS. 8A and 8B). The optimal myristic acid tail ((CO)—R²⁰, where R²⁰ is linear (C₁₃)alkyl; C₁₃H₃₁) in position 1 (group T) identified during Round 1 was retained in all Round 2 derivatives. Substitutions in position 6 (R⁵) included an imidazole group (Nim1; 911-M14), a naphthyl group (Nnap; 911-M15), a para-methoxybenzyl group (NphOMe; 911-M16), a 5-cyclohexylethyl group (NchexmS; 911-M17), a furfuryl group (Nfur; 911-M18), a para-fluorobenzyl group (Npfb; 911-M19), a cyclohexylmethyl group (Nchm; 911-M20), a thiophene group (Ntma; 911-M21), and a (±)-phenylethyl group (Npea; 911-M22). Structures were confirmed by ESI-MS and hydrophobicity was determined using MarvinSketch to calculate the distribution coefficient at pH 7.4 (Table 8).

TABLE 8
Physicochemical characterization of Round 2 derivatives.
Observed molecular weights determined by ESI-MS. cLogD_{7.4}
provided as a measure of hydrophobicity. cLogD_{7.4} =
calculated distribution coefficient at pH 7.4 ((Chemaxon.
MarvinSketch 19.17.0 was used for physicochemical
calculations. (https://www.chemaxon.com) (2019)).

| Compound | Expected m/z | Observed m/z | % yield | cLogD_{7.4} |
|---|---|---|---|---|
| 911-M14 | 1089.72 | 1090.12 | 7.6 | −4.40 |
| 911-M15 | 1107.70 | 1108.11 | 12.8 | −1.51 |
| 911-M16 | 1101.71 | 1102.13 | 29.2 | −2.67 |
| 911-M17 | 1091.76 | 1092.17 | 41.0 | −1.88 |
| 911-M18 | 1061.68 | 1062.07 | 14.4 | −3.47 |
| 911-M19 | 1089.69 | 1090.10 | 27.8 | −2.28 |
| 911-M20 | 1077.74 | 1078.07 | 14.5 | −2.30 |
| 911-M21 | 1077.65 | 1077.92 | 24.5 | −2.59 |
| 911-M22 | 1085.71 | 1087.04 | 26.1 | −2.00 |

Antifungal activity was determined for all derivatives against C. albicans and C. neoformans and is reported as MIC (Table 9). Mammalian cytotoxicity was determined against HepG2 liver cells and is reported as TD₅₀ (Table 9). These data indicate that none of the Round 2 derivatives had improved selectivity compared to 911-M1. Most compounds had similar antifungal activity to 911-M1, except for 911-M14 containing an imidazole group (Nim1), which had significantly diminished activity. Toxicity varied, but most compounds were comparable to 911-M1 with the exception of those containing cyclohexyl groups (911-M17, Nchexm; and 911-M20, Nchm), which were markedly more toxic. Within Round 2, compound 911-M18 had the best cytotoxicity profile, with a TD₅₀ of 151.96±8.04 μg/mL, which is slightly though not significantly better than 911-M1.

FIG. 9 shows a series of Round 2 hybrids that are being designed to test combinations of some of the promising modifications explored in rounds 1 and 2. FIG. 10 shows example round 3 derivatives that will be synthesized to explore modifications to the furfuryl group in position 5 (R⁴) followed by the propyl group in position 2 (R¹).

TABLE 9
Antifungal and cytotoxicity data for Round 2 derivatives.
MIC = minimum inhibitory concentration. TD₅₀ =
toxicity dose 50%. SR = selectivity ratio (SR = TD₅₀/MIC).

| | C. | C. | HepG2 | C. | C. |
|---|---|---|---|---|---|
| | albicans | neoformans | TD₅₀ | albicans | neoformans |
| Compound | (μg/mL) | (μg/mL) | (μg/mL) | SR | SR |
| 911-M14 | 25 | 6.25-12.5 | >200 | n/a | n/a |
| 911-M15 | 6.25 | 3.13-6.25 | 122.40 ± 21.27 | 20 | 20-39 |
| 911-M16 | 6.25 | 3.13-6.25 | 135.74 ± 39.60 | 22 | 22-43 |
| 911-M17 | 6.25 | 3.13-6.25 | 81.60 ± 5.97 | 13 | 13-26 |
| 911-M18 | 6.25 | 6.25 | 151.96 ± 8.04 | 24 | 24 |
| 911-M19 | 6.25 | 3.13 | 106.34 ± 34.83 | 17 | 34 |
| 911-M20 | 6.25 | 3.13 | 79.59 ± 5.12 | 13 | 25 |
| 911-M21 | 6.25 | 3.13-6.25 | 134.99 ± 52.15 | 22 | 43 |
| 911-M22 | 6.25 | 3.13 | 113.07 ± 41.85 | 18 | 36 |
| RMG 9-11 | 6.25 | 3.13 | 114 ± 5 | 18 | 36 |
| 911-M1 | 3.13-6.25 | 3.13 | 144.5 ± 22.6 | 23-46 | 46 |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A compound of the general formula, 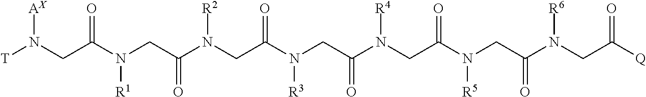
a protonated form thereof, a pharmaceutically acceptable salt thereof, or both,
wherein:
A^{X} is H or a linear or branched (C₆ to C₂₀)alkyl or a linear or branched (C₆ to C₂₀)alkenyl, wherein the alkyl or the alkenyl optionally includes a carbonyl group;
T is a linear or branched (C₆ to C₂₀)alkyl or a linear or branched (C₆ to C₂₀)alkenyl, wherein the alkyl or the alkenyl optionally includes a carbonyl group;
Q is a hydroxyl or NH₂; and
R¹, R², R³, R⁴, R⁵, and R⁶ are each independently 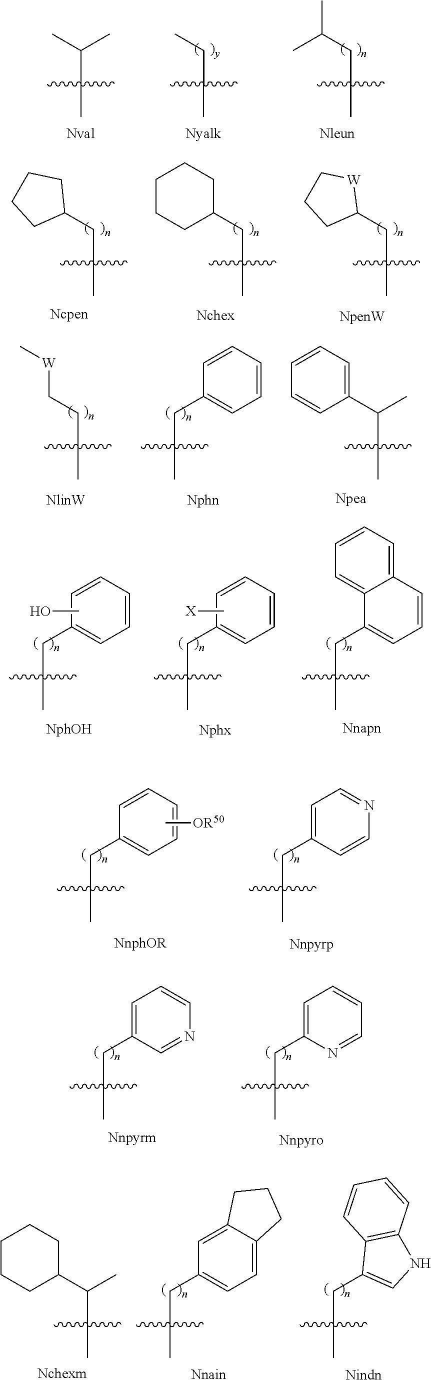 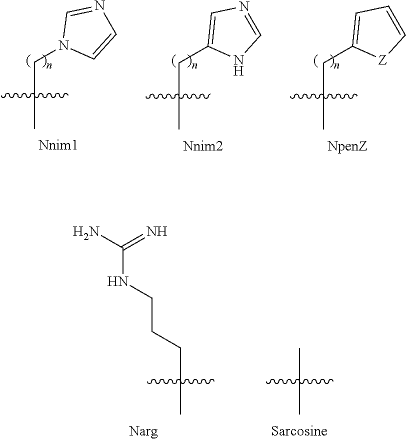
or an alkyl amine of the general formula R¹⁰NR¹¹R¹²R¹³,
wherein:
each n is independently (C₀ to C₃)alkylene;
each y is independently (C₀ to C₆)alkylene;
for each R¹, R², R³, R⁴, R⁵, and/or R⁶ that is NcpenW, each W is independently N, S, or O;
for each R¹, R², R³, R⁴, R⁵, and/or R⁶ that is NlinW, each W is independently N, S, or O;
for each R¹, R², R³, R⁴, R⁵, and/or R⁶ that is NphX, each X is independently F, Cl, Br, or I;
for each R¹, R², R³, R⁴, R⁵, and/or R⁶ that is NpenZ, each Z is independently S, or O;
for each R¹, R², R³, R⁴, R⁵, and/or R⁶ that is R¹⁰NR¹¹R¹²R¹³, each R¹⁰ is independently a linear (C₁-C₆)alkylene;
for each R¹, R², R³, R⁴, R⁵, and/or R⁶ that is NnphOR, each R⁵⁰ is independently (C₁ to C₃)alkyl; and
for each R¹, R², R³, R⁴, R⁵, and/or R⁶ that is R¹⁰NR¹¹R¹²R¹³, each R¹¹, each R¹², and each R¹³ are independently H or (C₁ to C₆)alkyl.

2. The compound of claim 1, wherein R¹, R², R³, R⁴, R⁵, and R⁶ are each independently

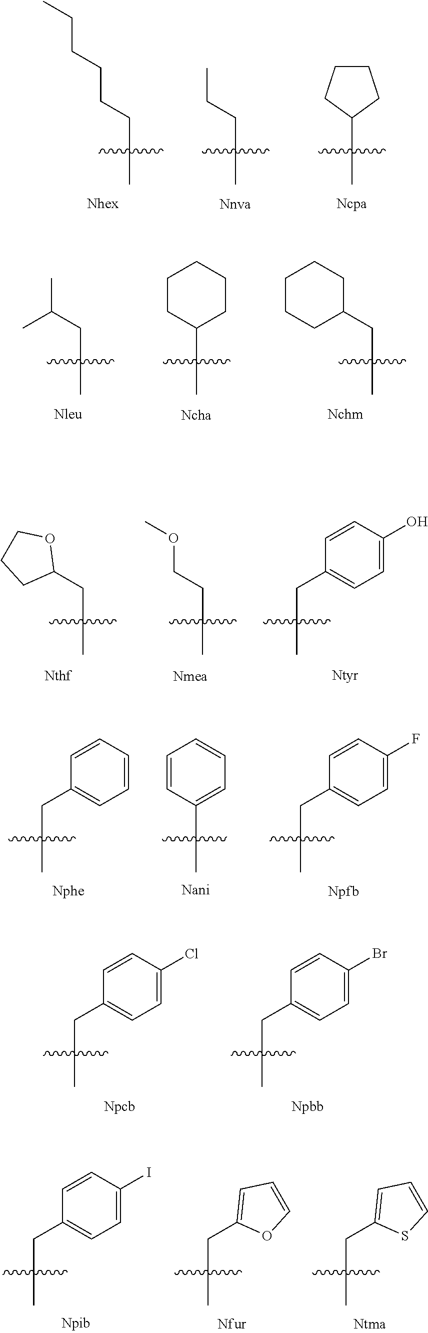

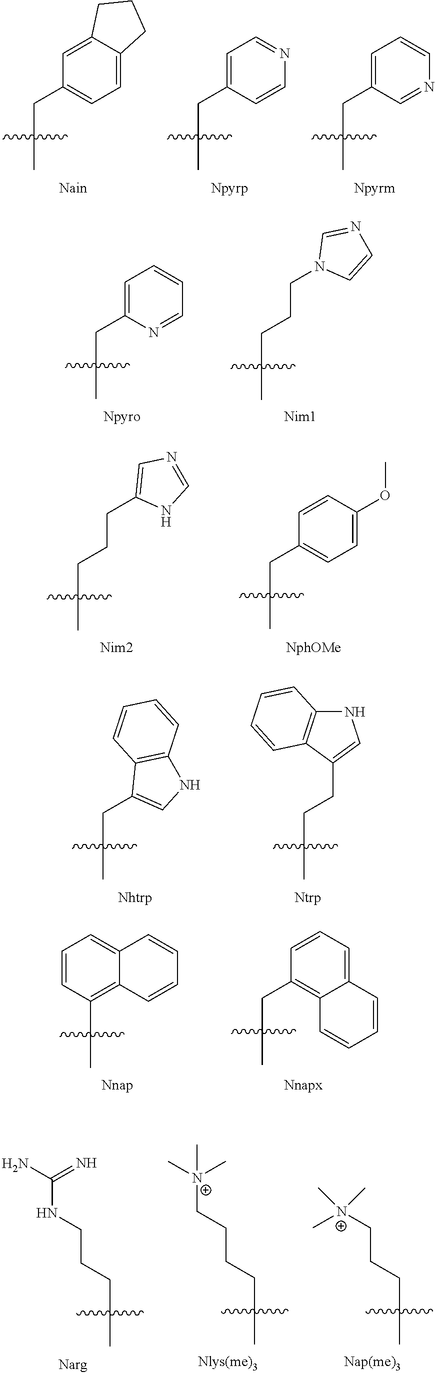

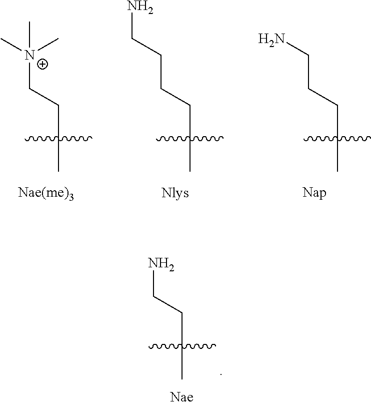

3. The compound of claim 1 wherein, T is a linear (C₁₃)alkyl; linear (C₁₅)alkyl; or (CO)R²⁰, wherein R²⁰ is linear (C₁₃)alkyl or linear (C₁₅)alkyl.

4. (canceled)

5. The compound of claim 1, wherein R¹¹, R¹², and R¹³ are methyl.

6. The compound of claim 1, wherein any one of R¹, R², R³, R⁴, R⁵, and R⁶ is Nnva; any one of R¹, R², R³, R⁴, R⁵, and is Npea; any one of R¹, R², R³, R⁴, R⁵e and R⁶ is Nlys; any one of R¹, R², R³, R⁴, R⁵, and R⁶ is Nfur; any one of R¹, R²R³, R⁴, R⁵, and R⁶ is Nphe; and any one of R¹, R², R³, R⁴, R⁵, and R⁶ is Nap.

7. The compound of claim 6, having the structure 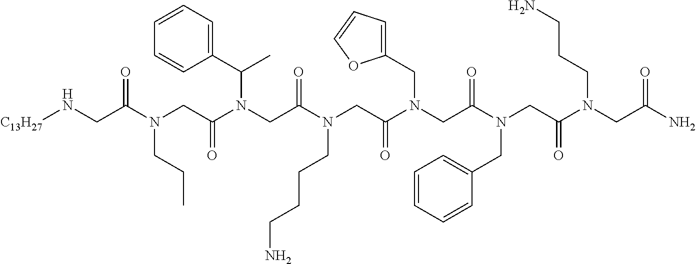
or a protonated form thereof.

8. A composition comprising:
a compound of the general formula, 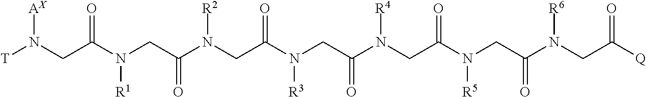
or a protonated form thereof, wherein:
A^{X} is H or a linear or branched (C₆ to C₂₀)alkyl or a linear or branched (C₆ to C₂₀)alkenyl, wherein the alkyl or the alkenyl optionally includes a carbonyl group;
T is a linear or branched (C₆ to C₂₀)alkyl or a linear or branched (C₆ to C₂₀)alkenyl, wherein the alkyl or the alkenyl optionally includes a carbonyl group;
Q is a hydroxyl or NH₂; and
R¹, R², R³, R⁴, R⁵, and R⁶ are each independently 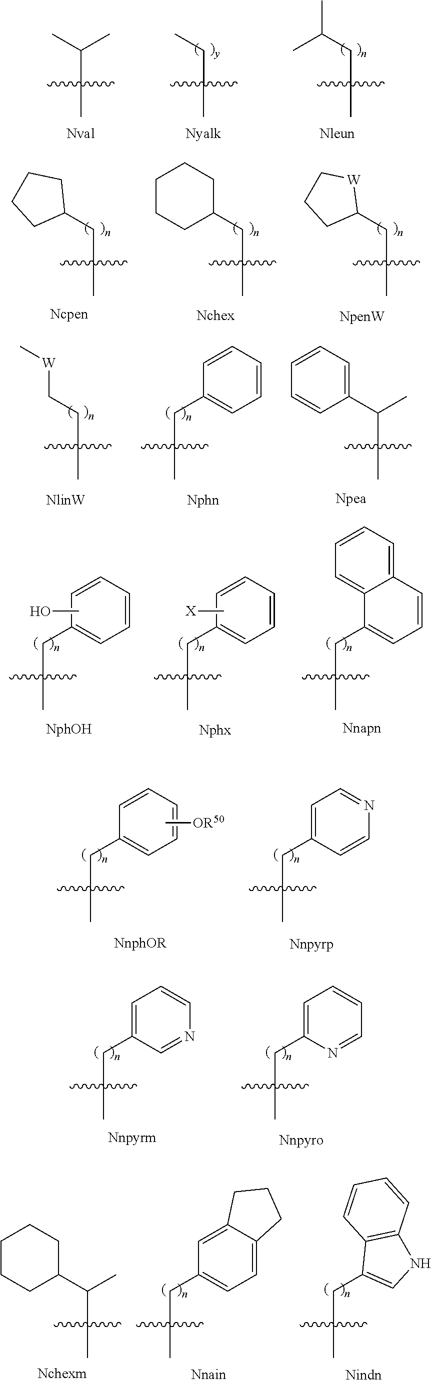 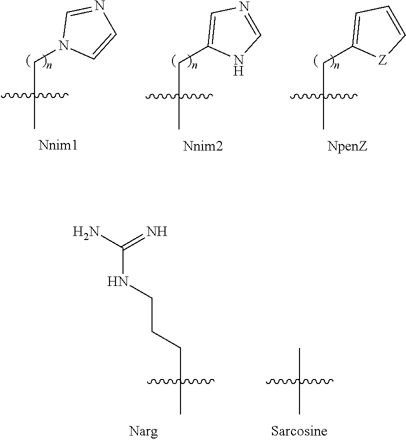
or an alkyl amine of the general formula R¹⁰NR¹¹R¹²R¹³,
wherein:
each n is independently (C₀-C₃)alkylene;
each y is independently (C₀-C₆)alkylene;
for each R¹, R², R³, R⁴, R⁵, and/or R⁶ that is NcpenW, each W is independently N, S, or O;
for each R¹, R², R³, R⁴, R⁵, and/or R⁶ that is NlinW, each W is independently N, S, or O;
for each R¹, R², R³, R⁴, R⁵, and/or R⁶ that is NphX, each X is independently F, Cl, Br, or I;
for each R¹, R², R³, R⁴, R⁵, and/or R⁶ that is NpenZ, each Z is independently S, or O;
for each R¹, R², R³, R⁴, R⁵, and/or R⁶ that is R¹⁰NR¹¹R¹²R¹³, each R¹⁰ is independently a linear (C₁-C₆)alkylene;
for each R¹, R², R³, R⁴, R⁵, and/or R⁶ that is NnphOR, each R⁵⁰ is independently (C₁ to C₃)alkyl; and
for each R¹, R², R³, R⁴, R⁵, and/or R⁶ that is R¹⁰NR¹¹R¹²R¹³, each R¹¹, each R¹², and each R¹³ are independently H or (C₁ to C₆)alkyl; and
a pharmaceutically acceptable carrier or fungicidal acceptable carrier.

9. The composition of claim 8, wherein R¹, R², R³, R⁴, R⁵, and R⁶ are each independently

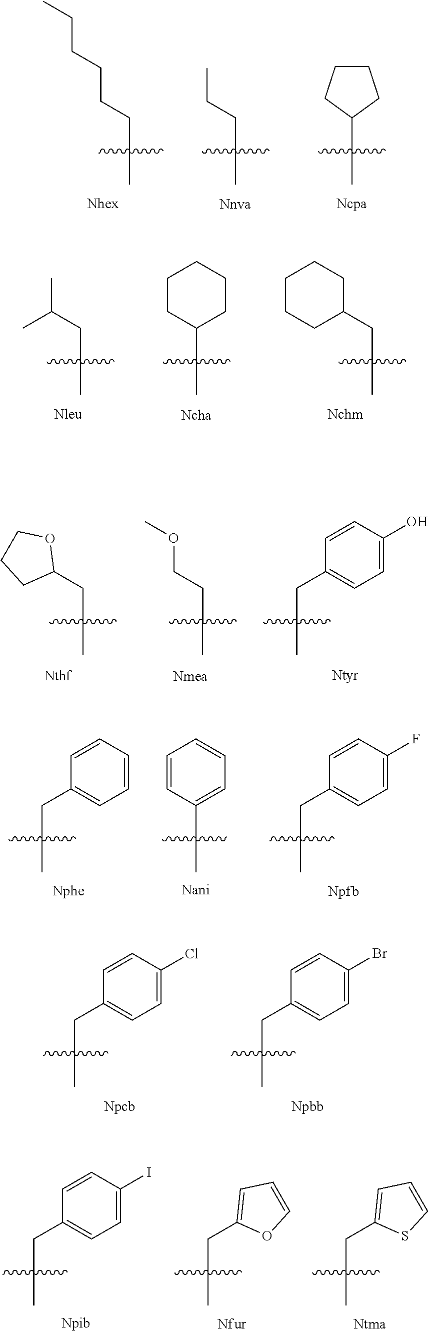

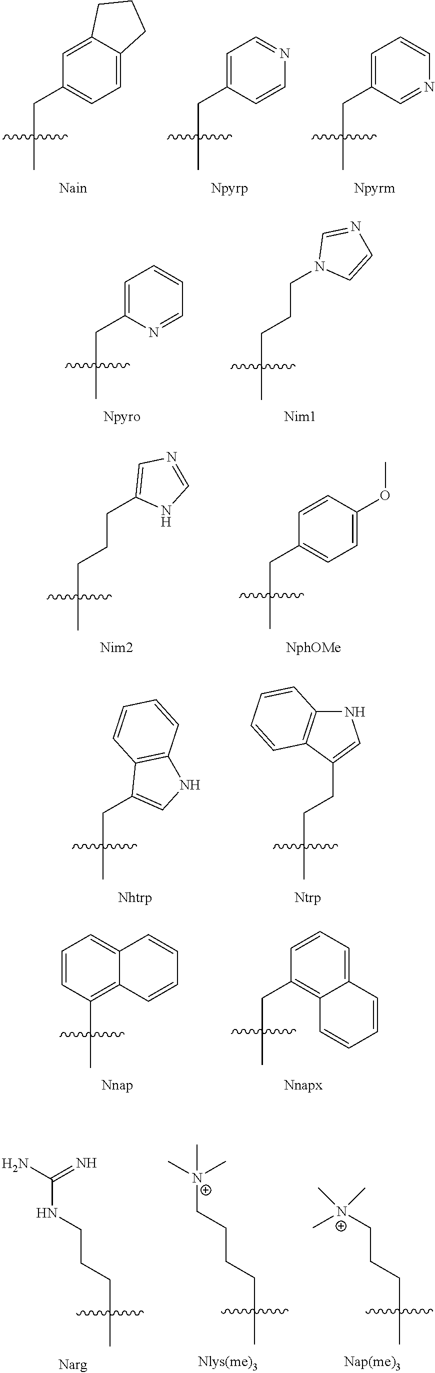

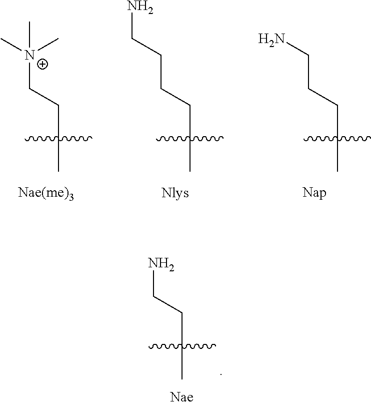

10. The composition of claim 8, wherein T is linear (C₁₃)alkyl; linear (C₁₅)alkyl; or (CO)R²⁰, wherein R²⁰ is linear (C₁₃)alkyl or linear (C₁₅)alkyl.

11. (canceled)

12. The composition of claim 8, wherein R¹¹, R¹², and R¹³ are methyl.

13. The composition of claim 8, wherein any one of R¹, R², R³, R⁴, R⁵, and R⁶ is Nnva; any one of R¹, R², R³, R⁴, R⁵, and is Npea; any one of R¹, R², R³, R⁴, R⁵, and R⁶ is Nlys; any one of R¹, R², R³, R⁴, R⁵, and R⁶ is Nfur; any one of R¹, R², R³, R⁴, R⁵, and R⁶ is Nphe; and any one of R¹, R², R³, R⁴, R⁵, and R⁶ is Nap.

14. The composition of claim 13, having the structure 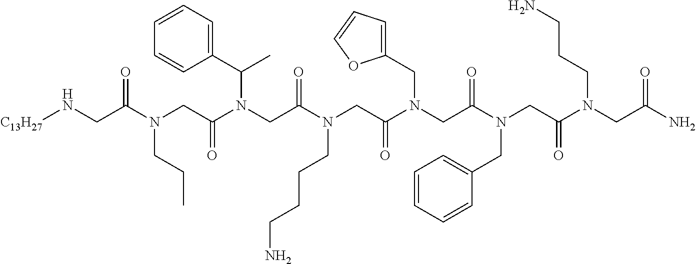
or a protonated form thereof.

15. A method comprising administering the composition of claim 8 to a subject, wherein the subject is a human or an animal.

16. (canceled)

17. The method of claim 15 wherein the method further comprises treating or preventing a fungal infection, or a biofilm produced from a fungal infection, or a combination thereof in the subject.

18. The method of claim 17 wherein the fungal infection comprises Candida albicans, Candida albicans, Candida auris, Candida tropicalis, Candida glabrata, Candida krusie, Candida parapsilosis, Rhodotorula mucilaginosa, Rhodotorula minuta, or Rhodotorula glutinis, Cryptococcus neoformans, or Cryptococcus gattii, or any combination thereof.

19-20. (canceled)

21. The method of claim 15 wherein the method further comprises treating or preventing a bacterial infection, or a biofilm produced from a bacterial infection, or a combination thereof, in the subject.

22-23. (canceled)

24. The method of claim 21 wherein the bacterial infection comprises tuberculosis, a gram-positive bacterium, or a gram-negative bacterium.

25-27. (canceled)

28. A method comprising administering the composition of claim 8 to a plant, a seed of a plant, or a soil the plant grows in.

29. The method of claim 28 wherein the method further comprises treating or preventing a fungal infection, or a biofilm produced from a fungal infection, or a combination thereof, in the plant.

30-32. (canceled)

33. The compound of claim 1 wherein a second minimum inhibitory concentration is no greater than a 200% of a first minimum inhibitory concentration, wherein the first minimum inhibitory concentration is evaluated without a serial gain of resistance assay against a microbe of interest, and the second minimum inhibitory concentration is evaluated after the serial gain of resistance assay against the microbe of interest.

34. (canceled)
